(19) United States
(12) Patent Application Publication
(10) Pub. No.: US 2012/0178158 A1
(43) Pub. Date: Jul. 12, 2012

(54) ADULT ANIMALS GENERATED FROM INDUCED PLURIPOTENT CELLS

(75) Inventor: Kristin K. Baldwin, La Jolla, CA (US)

(73) Assignee: The Scripps Research Institute, La Jolla (US)

(21) Appl. No.: 13/379,590

(22) PCT Filed: Jun. 23, 2010
(86) PCT No.: PCT/US10/39679

§ 371 Date: Mar. 16, 2012

Related U.S. Application Data
(60) Provisional application No. 61/269,412, filed on Jun. 23, 2009.
(60) Provisional application No. 61/230,062, filed on Jul. 30, 2009.

(51) Int. Cl. C12N 5/071

(52) U.S. Cl. 435/354; 435/377

(57) ABSTRACT

The present invention provides methods and compositions for generating and using induced pluripotent stem cells.

### CROSS-REFERENCE TO RELATED PATENT APPLICATIONS

The present application claims benefit of priority to U.S. Provisional Application No. 61/269,412, filed Jun. 23, 2009 and U.S. Provisional Application No. 61/230,062, filed Jul. 30, 2009, each of which are incorporated by reference.

### BACKGROUND OF THE INVENTION

Recent landmark experiments have shown that transient overexpression of a small number of transcription factors can reprogram differentiated cells into induced pluripotent stem (iPS) cells that resemble embryonic stem (ES) cells (Takahashi, K. et al., Cell 131:861-872 (2007); Takahashi, K. et al., Cell 126:663-676 (2006); Wernig, M. et al. Nature 448:318-324 (2007); Park, I. H. et al., Nat Protoc 3:1180-1186 (2008); Yu, J. et al. Science 318:1917-1920 (2007); Maherali, N. et al., Cell Stem Cell 1:55-70 (2007); Zhou, H. et al., Cell Stem Cell 4:381-384 (2009)). These iPS cells hold great promise for medicine because they have the potential to generate patient-specific cell types for cell replacement therapy and produce in vitro models of disease, without requiring embryonic tissues or oocytes (Ebert, A. D. et al., Nature 457:277-280 (2009); Park, I. H. et al., Cell 134:877-886 (2008); Dimos, J. T. et al., Science 321:1218-1221 (2008)). While current iPS cell lines can generate multiple cell types in vitro and produce viable chimeric mice, questions remain about their functional equivalence to ES cells. Importantly, current iPS cell lines have not produced full-term or adult mice in tetraploid complementation experiments. To date, mouse embryos produced exclusively from iPS cells fail to survive past embryonic day E14.5 (Wernig, M. et al. Nature 448:318-324 (2007); Hanna, J. et al., Cell 133:250-264 (2008)), a developmental stage at which many therapeutically important cell types, including brain glia, neural subtypes, kidney, bone marrow and aortic cells have yet to arise while other tissues remain immature (Kaufman, M. H., et al., The Anatomical Basis of Mouse Development. (Harcourt Brace and Company, London; 1999)). Whether this difference between iPS cells and pluripotent ES cells reflects intrinsic limitations of genetic reprogramming is not known.

### BRIEF SUMMARY OF THE INVENTION

The present invention provides methods for inducing full pluripotency in non-pluripotent animal cells, e.g., such that the induced full pluripotent cell lines have the capacity to generate live full term animals. In some embodiments, the method comprises,

introducing one or more transcription factor expression cassette(s) into non-pluripotent (e.g., non-embryonic) animal cells, which expression cassette(s) comprise a promoter operably linked to a polynucleotide encoding one or more transcription factors sufficient to induce pluripotency into the cells, where expression of the transcription factors is controlled by a tetracycline and/or doxycycline-inducible tetO regulatory element; and
introducing a transcriptional activator expression cassette comprising a promoter operably linked to a polynucleotide encoding a tetracycline and/or doxycycline responsive transcriptional activator, the transcriptional activator comprising a reverse tet repressor fused to a heterologous transactivation domain;
contacting the cells comprising the transcription factor expression cassette(s) and the transcriptional activator expression cassette with doxycycline, tetracycline, or a tetracycline analog; and
selecting cells that are pluripotent, thereby inducing pluripotency in non-pluripotent animal cells.

In some embodiments, the animal cell is a mouse cell. In some embodiments, the animal cell is a non-human animal cell. In some embodiments, the animal cell is a human cell.

In some embodiments, the cells are contacted with doxycycline, tetracycline, or a tetracycline analog for at least 13, 14, 15, 16, 17, 18, 19 or more days prior to the selecting step. In some embodiments, the cells are contacted with doxycycline, tetracycline, or a tetracycline analog for 13-30, 15-30, 17-30, 19-30, 13-50, 15-50, 17-50, or 19-50 days prior to the selecting step. In some embodiments the culture comprises a molecule (e.g. a protein or small molecule, e.g., under 1500 daltons) that maintains appropriate epigenetic marks (e.g., acetylation or methylation of histones and/or methylation of DNA in the Dlk1-Gtl2 (the “gtl2”) locus), allowing gene expression to occur that enhances or controls pluripotency or the ability to generate live offspring. In some embodiments the small molecule is valproic acid. In some embodiments the epigenetic marks comprise the Dlk1-Gtl2 imprinted gene locus. In some embodiments, the contacting step comprises contacting the cells with a histone deacetylation inhibitor. In some embodiments, the histone deacetylation inhibitor is valproic acid (VPA).

In some embodiments, the method comprises introducing one or more transcription factor expression cassette(s) into non-pluripotent animal cells, which expression cassette(s) comprise a promoter operably linked to a polynucleotide encoding one or more transcription factors sufficient to induce pluripotency into the cells, where expression of the transcription factors is controlled by an inducible element that can be induced by an inducer; and introducing a transcriptional activator expression cassette comprising a promoter operably linked to a polynucleotide encoding an inducer-responsive transcriptional activator; contacting the cells comprising the transcription factor expression cassette(s) and the transcriptional activator expression cassette with the inducer; contacting the cells with a chromatin modifier or histone deacetylase inhibitor; and selecting cells that are pluripotent, thereby inducing pluripotency in non-pluripotent animal cells.

In some embodiments, the cells are contacted with (1) the inducer and (2) the histone deacetylase inhibitor for at least 13 days prior to the selecting step. In some embodiments, the cells are contacted with (1) the inducer and (2) the histone deacetylase inhibitor for 13-30 days prior to the selecting step. In some embodiments, the cells are contacted with (1) the inducer and (2) the histone deacetylase inhibitor for 19-30 days prior to the selecting step.

In some embodiments, the histone deacetylation inhibitor is valproic acid.

In some embodiments, wherein the expression of the transcription factors is controlled by a tetracycline and/or doxycycline-inducible tetO regulatory element; and the method comprises introducing a transcriptional activator expression cassette comprising a promoter operably linked to a polynucleotide encoding a tetracycline and/or doxycycline responsive transcriptional activator, wherein the transcriptional activator comprises a reverse tet repressor fused to a heterologous transactivation domain. In some embodiments, the inducer is doxycycline, tetracycline, or a tetracycline analog.

In some embodiments, the heterologous transactivation domain comprises the fusion of two heterologous mammalian transactivation domains. In some embodiments, the two mammalian tranactivation domains are a NFκB p65 activation domain and an HSF1 activation domain. In some embodiments, the transactivation domain is rtTAM2.2

In some embodiments, the one or more transcription factors comprise at least a Sox polypeptide and an Oct3/4 polypeptide.

In some embodiments, the one or more transcription factors comprise Oct4, Sox2, Klf4, and c-Myc.

In some embodiments, the transcription factor expression cassette(s) and the transcriptional activator expression cassette are introduced as part of a viral vector. In some embodiments, the viral vector is a lentiviral vector or an adenoviral vector.

In some embodiments, the method further comprises injection of one or more selected cell lines into tetraploid blastocysts; and inserting the injected blastocysts into a uterus of a receptive non-human female animal. In some embodiments, the method further comprises obtaining from the female, progeny derived from the selected cell lines. In some embodiments, all of the tissues of the progeny are derived from the selected cell lines.

The present invention also provides an isolated animal (e.g., non-embryonic) cell, animal cell culture, or a transgenic non-human animal having cells comprising:

one or more transcription factor expression cassette(s), which expression cassette(s) comprise a promoter operably linked to a polynucleotide encoding one or more transcription factors sufficient to induce pluripotency into the cells, where expression of the transcription factors is controlled by a tetracycline and/or doxycycline-inducible tetO regulatory element; and
a transcriptional activator expression cassette comprising a promoter operably linked to a polynucleotide encoding a tetracycline and/or doxycycline responsive transcriptional activator, the transcriptional activator comprising a reverse tet repressor fused to a heterologous transactivation domain.

In some embodiments, the heterologous transactivation domain comprises the fusion of two heterologous mammalian transactivation domains.

In some embodiments, the two mammalian tranactivation domains are a NFκB p65 activation domain and an HSF1 activation domain. In some embodiments, the transactivation domain is rtTAM2.2.

In some embodiments, the one or more transcription factors comprise at least a Sox polypeptide and an Oct3/4 polypeptide.

In some embodiments, the one or more transcription factors comprise Oct3/4, Sox2, Klf4, and c-Myc.

In some embodiments, the animal is a mouse. In some embodiments, the animal is a non-human animal. In some embodiments, the animal is a human.

In some embodiments the culture comprises a molecule (e.g. a protein or small molecule, e.g., under 1500 daltons) that maintains appropriate epigenetic marks (e.g., acetylation or methylation of histones and/or methylation of DNA in the Dlk1-Gtl2 locus), allowing gene expression to occur that enhances or controls pluripotency or the ability to generate live offspring. In some embodiments the small molecule is a histone deacetylase inhibitor or chromatin modifier including but not limited to valproic acid. In some embodiments the epigenetic marks comprise genomic imprinting at the Dlk1-Gtl2 gene locus. In some embodiments, the culture comprises a histone deacetylase inhibitor. In some embodiments, the histone deacetylase inhibitor is valproic acid.

The present invention also provides methods for generating induced fully pluripotent cells capable of generating an adult animal. In some embodiments, the method comprises,

inducing pluripotency in a plurality of non-pluripotent (e.g., non-embryonic) animal cells to produced induced pluripotent cell lines;
inducing embryoid body formation from the induced pluripotent cell lines;
screening the embryoid bodies for expression of an adult-specific promoter;
selecting one or more cell lines that produce embryoid bodies that express the adult-specific promoter.

In some embodiments, the inducing pluripotency step lasts at least 13, 14, 15, 16, 17, 18, 19 days prior to the selecting step. In some embodiments, the inducing pluripotency step lasts for 13-30, 15-30, 17-30, 19-30, 13-50, 15-50, 17-50, or 19-50 days prior to the selecting step. In some embodiments, the inducing pluripotency step comprises contacting the cells with a histone deacetylation inhibitor. In some embodiments, the histone deacetylation inhibitor is valproic acid.

In some embodiments, the method further comprises injection of one or more selected cell lines into tetraploid blastocysts; and inserting the injected blastocysts into a uterus of a receptive female animal. In some embodiments, the method further comprises obtaining from the female, progeny derived from the selected cell lines. In some embodiments, all of the tissues of the progeny are derived from the selected cell lines.

In some embodiments, the animal is a mouse. In some embodiments, the animal is a non-human animal. In some embodiments, animal is a human.

In some embodiments, the pluripotent cell lines comprise at least one gene knockout or at least one recombinantly-introduced transgene (other than transgenes encoding iPSC-inducing transcription factors).

In some embodiments, the inducing step comprises introducing one or more transcription factors into the cells, thereby producing induced pluripotent stem cells. In some embodiments, the one or more transcription factors comprise at least a Sox polypeptide and an Oct3/4 polypeptide. In some embodiments, the one or more transcription factors comprise Oct4, Sox2, Klf4, and c-Myc.

In some embodiments, the induced pluripotent cell lines comprise an detectable marker expression cassette, the expression cassette comprising the adult-specific promoter operably linked to a reporter polynucleotide and the screening step comprises screening the embryoid bodies for production of the detectable marker polypeptide.

In some embodiments, the induced pluripotent cell lines comprise a recombinase expression cassette and a recombinase site expression cassette,

the recombinant expression cassette comprising an adult-specific promoter operably linked to a polynucleotide encoding a recombinase; and

the recombinase site expression cassette comprising:

a promoter operably linked to a first reporter polynucleotide; and

a second reporter polynucleotide,

wherein the first reporter polynucleotide is spanned by recombinase sites such that the promoter controls expression of the first reporter polynucleotide prior to contact of the recombinase to the recombinase site expression cassette and such that the promoter controls expression of the second reporter polynucleotide upon contact of the recombinase-initiated recombination of the recombinase site expression cassette.

In some embodiments, the recombinase is Cre and the recombinase sites are lox sites. In some embodiments, the reporter polynucleotide(s) is a fluorescent protein. In some embodiments, the adult specific promoter is selected from the group consisting of a promoter that is expressed in olfactory bulb mitral cells, an olfactory-specific promoter, a Pcdh21 promoter, a neuron specific promoter, a neuron specific promoter, and a glial-specific promoter.

In some embodiments, the one or more transcription factors are introduced into the cells by introducing one or more iPSC expression cassette into the cells, wherein the iPSC expression cassette comprises a promoter operably linked to polynucleotide encoding one or more of the one or more transcription factors. In some embodiments, the promoter in the one or more iPSC expression cassettes is a promoter that is activated when bound by a reverse tetracycline transactivator (rtTA) and contacted by doxycycline, tetracycline, or a tetracycline analog. In some embodiments, the rtTA is rtTAM2.2.

In some embodiments, the promoter is the tetO promoter.

In some embodiments, one iPSC expression cassette is introduced into the cells and the iPSC expression cassette is polycistronic and encodes more than one transcription factor for inducing pluripotency.

The present invention also provides an isolated induced fully pluripotent (e.g., non-embryonic) animal cell comprising:

a. a recombinase expression cassette and a recombinase site expression cassette,

the recombinase expression cassette comprising an adult-specific promoter operably linked to a polynucleotide encoding a recombinase; and

the recombinase site expression cassette comprising:

a promoter operably linked to a first reporter polynucleotide; and

a second reporter polynucleotide,

wherein the first reporter polynucleotide is spanned by recombinase sites such that the promoter controls expression of the first reporter polynucleotide prior to contact of the recombinase to the recombinase site expression cassette and such that the promoter controls expression of the second reporter polynucleotide upon contact of the recombinase-initiated recombination of the recombinase site expression cassette; and
b. one or more iPSC expression cassette comprising a promoter operably linked to a polynucleotide encoding one or more transcription factors, wherein expression of all of the one or more transcription factors is sufficient to induce pluripotency in a non-pluripotent cell.

In some embodiments, the cell is a mouse cell. In some embodiments, the cell is a non-human animal cell. In some embodiments, the cell is a human cell.

In some embodiments, the cell comprises at least one gene knockout or at least one recombinantly-introduced transgene (other than transgenes encoding iPSC-inducing transcription factors). In some embodiments, the one or more transcription factors comprise at least a Sox polypeptide and an Oct3/4 polypeptide. In some embodiments, the one or more transcription factors comprise Oct4, Sox2, Klf4, and c-Myc.

In some embodiments, the recombinase is Cre and the recombinase sites are lox sites.

In some embodiments, the reporter polynucleotide(s) is a fluorescent protein.

In some embodiments, the adult specific promoter is selected from the group consisting of a promoter that is expressed in olfactory bulb mitral cells, an olfactory-specific promoter, a Pcdh21 promoter, a neuron-specific promoter and a glial-specific promoter.

In some embodiments, the promoter in the one or more iPSC expression cassettes is a promoter that is activated when bound by a reverse tetracycline transactivator (rtTA) and contacted by doxycycline, tetracycline, or a tetracycline analog. In some embodiments, the rtTA is rtTAM2.2. In some embodiments, the promoter in the one or more iPSC expression cassettes is the tetO promoter.

In some embodiments, the cell comprises one iPSC expression cassette, which is polycistronic and encodes the one or more transcription factors.

The present invention also provides a method for inducing full pluripotency in non-pluripotent (e.g., non-embryonic) animal cells, the method comprising,

introducing one or more transcription factor expression cassette(s) into non-pluripotent animal cells, which expression cassette(s) comprise a promoter operably linked to a polynucleotide encoding one or more transcription factors sufficient to induce pluripotency into the cells, wherein the expression cassettes are inserted into the genome of the cell in no more than 1, 2, or 3 copies, and wherein the transcription factor expression cassettes are under control of an operator responsive to a transcriptional activator; and
introducing a transcriptional activator expression cassette comprising a promoter operably linked to a polynucleotide encoding the transcriptional activator, wherein the transcriptional activator activates expression from the transcription factor expression cassettes more than if a rTTam2 transcriptional activator were used;
inducing activation of the transcriptional activator, if necessary; and
selecting cells that are pluripotent, thereby inducing pluripotency in non-pluripotent animal cells.

In some embodiments, the inducing step lasts at least 13, 14, 15, 16, 17, 18, 19 days prior to the selecting step. In some embodiments, the inducing step lasts for 13-30, 15-30, 17-30, 19-30, 13-50, 15-50, 17-50, or 19-50 days prior to the selecting step. In some embodiments, the inducing step comprises contacting the cells with a chromatin modifier or histone deacetylation inhibitor. In some embodiments, the histone deacetylation inhibitor is valproic acid.

In some embodiments, the heterologous transactivation domain comprises the fusion of two heterologous mammalian transactivation domains. In some embodiments, the two mammalian transactivation domains are a NFκB p65 activation domain and an HSF1 activation domain. In some embodiments, the transactivation domain is rtTAM2.2

In some embodiments, the one or more transcription factors comprise at least a Sox polypeptide and an Oct3/4 polypeptide.

In some embodiments, the one or more transcription factors comprise Oct4, Sox2, Klf4, and c-Myc.

In some embodiments, the transcription factor expression cassette(s) and the transcriptional activator expression cassette are introduced as part of a viral vector. In some embodiments, the viral vector is a lentiviral vector or an adenoviral vector.

In some embodiments, the method further comprises injection of one or more selected cell lines into tetraploid blastocysts; and inserting the injected blastocysts into a uterus of a receptive female animal. In some embodiments, the method further comprises obtaining from the female, progeny derived from the selected cell lines. In some embodiments, all of the tissues of the progeny are derived from the selected cell lines.

In some embodiments, the animal is a mouse. In some embodiments, the animal is a non-human animal. In some embodiments, the animal is a human.

The present invention also provides an isolated (e.g., non-embryonic) animal cell, animal cell culture, or a transgenic animal having cells comprising:

one or more transcription factor expression cassette(s) into non-pluripotent animal cells, which expression cassette(s) comprise a promoter operably linked to a polynucleotide encoding one or more transcription factors sufficient to induce pluripotency into the cells, where expression of the transcription factors is controlled by a tetracycline and/or doxycycline-inducible tetO regulatory element; and
a transcriptional activator expression cassette comprising a promoter operably linked to a polynucleotide encoding a tetracycline and/or doxycycline responsive transcriptional activator, the transcriptional activator comprising a reverse tet repressor fused to a heterologous transactivation domain.

In some embodiments, the heterologous transactivation domain comprises the fusion of two heterologous mammalian transactivation domains.

In some embodiments, the two mammalian tranactivation domains are a NFκB p65 activation domain and an HSF1 activation domain. In some embodiments, the transactivation domain is rtTAM2.2.

In some embodiments, the one or more transcription factors comprise at least a Sox polypeptide and an Oct3/4 polypeptide.

In some embodiments, the one or more transcription factors comprise Oct4, Sox2, Klf4, and c-Myc.

In some embodiments, the animal is a mouse. In some embodiments, the animal is a non-human animal. In some embodiments, the animal is a human.

The present invention provides for isolated non-embryonic animal cell or cell line or cell culture, wherein the cell is capable of generating an adult animal in a tetraploid complementation assay, i.e., they are fully pluripotent. In some embodiments, the animal is a mouse. In some embodiments, the animal is a non-human animal. In some embodiments, the animal is a human. In some embodiments, the cells have an appropriate imprinting at the Dlk1-Gtl2 locus to allow for expression of RNA from the locus. In some embodiments, the Dlk1-Gtl2 locus is hemimethylated and/or comprises acetylated histones.

The present invention also provides methods of generating adult animals from induced pluripotent cells comprising inducing non-pluripotent cells to pluripotency, contacting the cells with a chromatin modifier or histone deacetylase inhibitor (including but not limited to valproic acid), and performing a tetraploid complementation assay (e.g., injecting the cells into tetraploid blastocysts, inserting the resulting cells into the uterus of a receptive female animal, and obtaining progeny derived from the inserted cells. The histone deacetylase inhibitor used, for example, can be used for a sufficient time, and in sufficient amount, to improve efficiency of the cells in a tetraploid complementation assay.

Other embodiments of the invention will be clear from the remainder of this document.

### DEFINITIONS

An “Oct polypeptide” refers to any of the naturally-occurring members of Octomer family of transcription factors, or variants thereof that maintain transcription factor activity, similar (within at least 50%, 80%, or 90% activity) compared to the closest related naturally occurring family member, or polypeptides comprising at least the DNA-binding domain of the naturally occurring family member, and optionally comprising a transcriptional activation domain. Exemplary Oct polypeptides include, e.g., Oct3/4 (referred to herein as “Oct4”), which contains the POU domain. See, Ryan, A. K. & Rosenfeld, M. G. Genes Dev. 11, 1207-1225 (1997). In some embodiments, variants have at least 90% amino acid sequence identity across their whole sequence compared to a naturally occurring Oct polypeptide family member such as to those listed above or such as listed in Genbank accession number NP_{—}002692.2 (human Oct4) or NP_{—}038661.1 (mouse Oct4).

A “Klf polypeptide” refers to any of the naturally-occurring members of the family of Krüppel-like factors (Klfs), zinc-finger proteins that contain amino acid sequences similar to those of the Drosophila embryonic pattern regulator Krüppel, or variants of the naturally-occurring members that maintain transcription factor activity similar (within at least 50%, 80%, or 90% activity) compared to the closest related naturally occurring family member, or polypeptides comprising at least the DNA-binding domain of the naturally occurring family member, and optionally comprising a transcriptional activation domain. See, Dang, D. T., Pevsner, J. & Yang, V. W. Cell Biol. 32, 1103-1121 (2000). Exemplary Klf family members include, e.g., Klf1, Klf4, and Klf5, each of which have been shown to be able to replace each other to result in iPS cells. See, Nakagawa, et al., Nature Biotechnology 26:101-106 (2007). In some embodiments, variants have at least 90% amino acid sequence identity across their whole sequence compared to a naturally occurring Klf polypeptide family member such as to those listed above or such as listed in Genbank accession number CAX16088 (mouse Klf4) or CAX14962 (human Klf4). To the extent a KLF polypeptide is described herein, it can be replaced with an Essrb. Thus, it is intended that for each Klf polypeptide embodiment described herein is equally described for use of Essrb in the place of a Klf4 polypeptide.

A “Myc polypeptide” refers any of the naturally-occurring members of the Myc family (see, e.g., Adhikary, S. & Eilers, M. Nat. Rev. Mol. Cell Biol. 6:635-645 (2005)), or variants thereof that maintain transcription factor activity similar (within at least 50%, 80%, or 90% activity) compared to the closest related naturally occurring family member, or polypeptides comprising at least the DNA-binding domain of the naturally occurring family member, and optionally comprising a transcriptional activation domain. Exemplary Myc polypeptides include, e.g., c-Myc, N-Myc and L-Myc. In some embodiments, variants have at least 90% amino acid sequence identity across their whole sequence compared to a naturally occurring Myc polypeptide family member such as to those listed above or such as listed in Genbank accession number CAA25015 (human Myc).

A “Sox polypeptide” refers to any of the naturally-occurring members of the SRY-related HMG-box (Sox) transcription factors, characterized by the presence of the high-mobility group (HMG) domain, or variants thereof that maintain transcription factor activity similar (within at least 50%, 80%, or 90% activity) compared to the closest related naturally occurring family member, or polypeptides comprising at least the DNA-binding domain of the naturally occurring family member, and optionally comprising a transcriptional activation domain. See, e.g., Dang, D. T., et al., Int. J. Biochem. Cell Biol. 32:1103-1121 (2000). Exemplary Sox polypeptides include, e.g., Sox1, Sox2, Sox3, Sox15, or Sox18, each of which have been shown to be able to replace each other to result in iPS cells. See, Nakagawa, et al., Nature Biotechnology 26:101-106 (2007). In some embodiments, variants have at least 90% amino acid sequence identity across their whole sequence compared to a naturally occurring Sox polypeptide family member such as to those listed above or such as listed in Genbank accession number CAA83435 (human Sox2).

The term “pluripotent” or “pluripotency” refers to cells with the ability to give rise to progeny that can undergo differentiation, under the appropriate conditions (e.g., a tetraploid complementation assay), into cell types that collectively demonstrate characteristics associated with cell lineages from all of the three germinal layers (endoderm, mesoderm, and ectoderm). Pluripotent stem cells can contribute to many or all tissues of a prenatal, postnatal or adult animal. A standard art-accepted test, such as the ability to form a teratoma in 8-12 week old SCID mice, can be used to establish the pluripotency of a cell population, however identification of various pluripotent stem cell characteristics can also be used to detect pluripotent cells. The gold standard test for pluripotency is generation of an animal derived entirely from a pluripotent cell line. This level of pluripotency may be termed “full pluripotency” and cells lines with this property may be termed “fully pluripotent”. Previously generated iPS cell lines failed tests of full pluripotency indicating that they could not properly generate all cell types in an organism.

“Pluripotent stem cell characteristics” refer to characteristics of a cell that distinguish pluripotent stem cells from other cells. The ability to give rise to progeny that can undergo differentiation, under the appropriate conditions, into cell types that collectively demonstrate characteristics associated with cell lineages from all of the three germinal layers (endoderm, mesoderm, and ectoderm) is a pluripotent stem cell characteristic. Expression or non-expression of certain combinations of molecular markers are also pluripotent stem cell characteristics. For example, human pluripotent stem cells express at least some, and optionally all, of the markers from the following non-limiting list: SSEA-3, SSEA-4, TRA-1-60, TRA-1-81, TRA-2-49/6E, ALP, Sox2, E-cadherin, UTF-1, Oct4, Rex1, and Nanog. Cell morphologies associated with pluripotent stem cells are also pluripotent stem cell characteristics.

“Expression cassette” refers to a polynucleotide comprising a promoter or other regulatory sequence operably linked to a nucleotide sequence to be transcribed, optionally encoding a protein.

The terms “promoter” and “expression control sequence” are used herein to refer to an array of nucleic acid control sequences that direct transcription of a nucleic acid. As used herein, a promoter includes necessary nucleic acid sequences near the start site of transcription, such as, in the case of a polymerase II type promoter, a TATA element. A promoter also optionally includes distal enhancer or repressor elements, which can be located as much as several thousand base pairs from the start site of transcription. Promoters include constitutive and inducible promoters. A “constitutive” promoter is a promoter that is active under most environmental and developmental conditions. An “inducible” promoter is a promoter that is active under environmental or developmental regulation. The term “operably linked” refers to a functional linkage between a nucleic acid expression control sequence (such as a promoter, or array of transcription factor binding sites) and a second nucleic acid sequence, wherein the expression control sequence directs transcription of the nucleic acid corresponding to the second sequence.

A “heterologous sequence” or a “heterologous nucleic acid”, as used herein, is one that originates from a source foreign to the particular host cell, or, if from the same source, is modified from its original form. Thus, a heterologous expression cassette in a cell is an expression cassette that is not endogenous to the particular host cell, for example by being linked to nucleotide sequences from an expression vector rather than chromosomal DNA, being linked to a heterologous promoter, being linked to a reporter gene, etc.

The terms “nucleic acid” and “polynucleotide” are used interchangeably herein to refer to deoxyribonucleotides or ribonucleotides and polymers thereof in either single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

Unless otherwise indicated, a particular nucleic acid sequence also encompasses conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); Rossolini et al., Mol. Cell. Probes 8:91-98 (1994)).

### BRIEF DESCRIPTION OF THE DRAWINGS

FIG. 1|Generation of iPSCs. a, Genetic marking strategy. Z/EG cells express β-geo. Rare neurons express Pcdh21/Cre causing GFP expression. b, Drug inducible reprogramming. Reprogramming factors (RFs-Oct4, Sox2, cMyc or Klf4) are controlled by the dox-inducible promoter (pTRE). The rtTAM2.2 is constitutive (pUbC; Ubiquitin promoter). c, Reprogramming timeline α-axis: days post-transduction). Dox and VPA treatment began on day 4 and ended on day 23. P, passage. d, Brightfield images of iMZ lines and ESCs (Top row). Immunofluorescence staining (second and third row) of iMZ lines for pluripotency markers Nanog (Green), S SEA-1 (Red) and nuclei (DAPI-Blue). e, iMZ iPSC-derived embryoid bodies contain GFP positive cells. Scale bars: 100 μm.

FIG. 2|Adult mice derived from iMZ cell lines. a, (from left) iPS mice (postnatal day p6) exhibit pigmented skin in comparison to CD-1 mice. iPS mice (postnatal day p10) and age matched albino CD-1 pups. Adult iMZ-9 iPS mouse (4 weeks) is morphologically normal. Germline transmission by 12-week iMZ-9 mouse (left) shown with 2-week old progeny (agouti) and mother (white). b, Tissue sections from a p10 Pcdh21/Cre-Z/EG mouse (top), iPS mice derived from iMZ-9 and iMZ-15 cell lines (middle) and wild type mouse (bottom) stain positive for β-geo with X-gal (Blue). Scale bar, 100 μm. c, Contribution to the olfactory bulbs of p10 chimeric mice (imZ-9, iMZ-11) and an iMZ-9 iPS mouse. Sections were stained for GFP (Green, mitral cells), β-galactosidase (Red, Z/EG cells) and nuclei (blue, TOTO-3). Scale bar, 100 μm.

FIG. 3|Genetic analysis of iPS mice. a, PCR assay of genomic DNA for the Cre (left) and Z/EG (right) genetic insertions in iMZ iPSCs and mice. Positive control(+) is Pcdh21/Cre-Z/EG tail DNA. Negative control (−) (no DNA) and ESCs (ES) were negative. b, Southern blots of genomic DNA from iMZ cell lines and an iMZ-9 iPS mouse show similar patterns of insertions. Probes were coding sequences of the Oct4 (left) or rtTAM2.2 (right) genes. Pcdh21/Cre mice and wild type ESCs are controls. Endogenous bands (*). c, Microsatellite PCR assay for tetraploid cells. Band size distinguishes iMZ cells from tetraploid host strains (C57BL/6J-Tyr^{c-2J} and BALB/cByJ). Left: DNA titration curve demonstrates 5% detection limit. Right: Analysis of DNA from tissues derived from an iPS mouse (IMZ-9). T: thymus, L: liver, Sk: skin, B: brain, Sp: spleen, H: heart, K: kidney. iPSCs: iMZ-9. C57: C57BL/6J-Tyr^{c-2J}. Blb: BALB/cByJ. MW: molecular weight marker. d, Albino allele PCR assay. Left: Albino Tyr^{c-2J} allele PCR assay can detect 0.5% tetraploid cell DNA diluted into iMZ DNA. i: iMZ-9, (−): H2O control, C: CD-1. The C57BL/6J-Tyr^{c-2J} allele is expected in 75% of tetraploid blastocysts. Right: MW, Tail DNA from different pups derived from iMZ iPS lines as noted, (−): H2O control, (+): C57BL/6J-Tyr^{c-2J}/DNA.

Table 1|Summary of blastocyst injections. All iPS cell lines that were tested are listed and those that contributed to diploid chimeras contain a Y in the second column (2n Chim.). Other lines were not tested. The number of individual blastocysts injected for each cell line (Blasts inj.) is shown. Pregnant dams were either dissected at E16.5 or E17.5 for analysis of tissues, or Cesaerean sections and cross fostering was performed at E18.5 as noted in column four. The number of live embryos for dissection or live pups at C-section is in columns five and six. The number of mice surviving after cross fostering is in column seven. Percentage of blasts injected as in parentheses.

### DETAILED DESCRIPTION

### I. Introduction

The present invention is based in part on the surprising discovery that induced pluripotent (e.g., non-embryonic) stem cells (iPSC) can be used to generate complete animals using the tetraploid complementation assay. The data presented herein is the first report of the generation of animals via tetraploid complementation from iPSCs. Thus, iPSCs generated and selected by the methods of the invention can be introduced into tetraploid blastocysts and subsequently introduced into a female animal to produce progeny whose cells are entirely derived from the iPSCs.

Tetraploid complementation assays/methods are known in the art. See, e.g., U.S. Pat. No. 6,492,575 and U.S. Pat. No. 6,784,336.

The data provided herein illustrates several useful lessons for generating iPSC lines from which a large number of independent lines are capable of generating live animals derived entirely from iPSCs.

II. Screens for Identifying iPSCs Likely Capable of Generating Animals in a Tetraploid Complementation Assay

In some embodiments, iPSCs (induced to pluripotency in any way) are cultured and a portion of such cells are induced to form embryoid bodies, wherein some cells in the embryoid bodies begin a differentiation process. By screening embryoid bodies derived from iPSCs for expression of an adult-specific promoter, one can pre-select iPSCs that are more prone to be capable of developing into whole animals in a tetraploid complementation assay/method than an iPSC selected randomly.

It is believed that any number of adult-specific promoters can be used according to this selection method. Adult-specific promoters are promoters that are expressed in adult tissues but are not expressed in embryo development. Thus, if an embryoid body is capable of expression from an adult-specific promoter, it is more likely that the iPSCs will be able to form all of the adult tissue types required to complete embryogenesis. Exemplary adult-specific promoters include, but are not limited to, promoters specific for a cell type that arises in development after stage E14 including but not limited to a neuron specific promoter, a glial-specific promoter (e.g., glial fibrillary acidic protein (GFAP)), or a promoter that is expressed in olfactory bulb mitral cells, e.g., an olfactory-specific promoter, e.g., a Pcdh21 promoter.

Expression from the adult-specific promoter can be detected by any method convenient. In some embodiments, prior or after induction of pluripotency, an expression cassette comprising the adult-specific promoter operably linked to a reporter polynucleotide is introduced into the cells. A reporter polynucleotide can be any polynucleotide that allows for efficient detection of expression. In some embodiments, the reporter polynucleotide will encode a detectable marker polypeptide, i.e., a polypeptide whose expression is readily detected in an embryoid body. For example, the reporter polypeptide can be a fluorescent protein (e.g., GFP) or a protein that is otherwise readily detectable, including but not limited to, proteins that emit a signal or are readily detectable by altering a substrate that, when modified, emits a signal.

In some embodiments, the adult-specific promoter is operably linked to a polynucleotide encoding a recombinase and is introduced into the cells with a second expression cassette comprising a first and second reporter polynucleotide, wherein a promoter (optionally a constitutive promoter) is operably linked to the first reporter polynucleotide, wherein the first reporter polynucleotide is spanned by recombinase recognition sites such that, when the expression cassette is contacted to a recombinase, the expression cassette is recombined such that the promoter is operably linked to the second reporter polynucleotide. Said another way, prior to contact with the recombinase, the promoter controls expression from the first polynucleotide whereas following contact with the recombinase, the promoter controls the expression from the second polynucleotide. This arrangement allows for confirmation of introduction of the second expression cassette (by monitoring expression of the first reporter polynucleotide) and also allows for monitoring of expression of the adult-specific promoter because cells in which the adult-specific promoter is expressed have a recombined second expression cassette resulting in expression of the second reporter polynucleotide.

A recombinase catalyzes a recombination reaction between specific recognition sequences. Recombination sites typically have an orientation. In other words, they are not perfect palindromes. In some aspects, the orientation of the recognition sequences in relation to each other determines what recombination event takes place. The recombination sites may be in two different orientations: parallel (same direction) or opposite. When the recombination sites are in an opposite orientation to each other, then the recombination event catalyzed by the recombinase is an inversion. When the recombination sites are in a parallel orientation, then any intervening sequence is excised. The reaction can often leave a single recombination site in the genome following excision. In some embodiments, it is this second orientation that is used in the methods of the invention to excise the first reporter polynucleotide.

One recombination system is the Cre-lox system. In the Cre-lox system, the recognition sequences are referred to as “lox sites” and the recombinase is referred to as “Cre”. When lox sites are in parallel orientation (i.e., in the same direction), then Cre catalyzes a deletion of the intervening polynucleotide sequence. When lox sites are in the opposite orientation, the Cre recombinase catalyzes an inversion of the intervening polynucleotide sequence. This system has been described in various host cells, including Saccharomyces cerevisiae (Sauer, B., Mol Cell Biol. 7:2087-2096 (1987)); mammalian cells (Sauer, B. et al., Proc. Natl Acad. Sci. USA 85:5166-5170 (1988); Sauer, B. et al., Nucleic Acids Res. 17:147-161 (1989)). Use of the Cre-lox recombinase system is also described in, e.g., U.S. Pat. No. 5,527,695 and PCT application No. WO 93/01283. Several different lox sites are known, including lox511 (Hoess R. et al., Nucleic Acids Res. 14:2287-2300 (1986)), lox66, lox71, lox76, lox75, lox43, lox44 (Albert H. et al., Plant J. 7(4): 649-659 (1995)).

Several other recombination systems are also suitable for use in the invention. These include, for example, the FLP/FRT system of yeast (Lyznik, L. A. et al., Nucleic Acids Res. 24(19):3784-9 (1996)), the Gin recombinase of phage Mu (Crisona, N. J. et al., J. Mol. Biol. 243(3):437-57 (1994)), the Pin recombinase of E. coli (see, e.g., Kutsukake K, et. al., Gene 34(2-3):343-50 (1985)), the PinB, PinD and PinF from Shigella (Tominaga A et al., J. Bacteriol. 173(13):4079-87 (1991)), the R/RS system of the pSRi plasmid (Araki, H. et al., J. Mol. Biol. 225(1):25-37 (1992)), recombination systems in theta-replicating bacteria (Alonso, et al., Ann. Rev. Biochem. 66:437-474 (1997) and the shufflon systems found in some prokaryotes (Komano, Ann. Rev. Genetics Res. Microbiol. 150(9-10):641-51 (1999). Other recombination systems include the integrase family of recombinases (Grainge, et al., Molec. Microbiol. 33(3):449-56 (1999); Gopaul et al., Curr. Opin. Struct. Biol. 9(1):14-20 (1999); Yang, et al., Structure 5(11):1401-6 (1997)).

In some embodiments, one or more of the above-described expression cassettes are used in combination with one or more “iPSC” expression cassettes, i.e., an expression cassette encoding one or more transcription factors for inducing pluripotency as described further below.

### III. Methods of Inducing Pluripotency in Non Pluripotent Cells

To date, a wide variety of methods for generating iPSCs have been developed and such methods can generally be applied to induce pluripotency in non-pluripotent cells according to the present invention, e.g., using the screening methods described herein. However, in some embodiments, the methods described herein, including but not limited to induction of iPSCs using inducible expression of transcription factors from viral vectors, specific gene expression regulators, etc., will be used. In some embodiments, the methods of inducing iPSCs will be optimized by using the induction timing and/or histone deacetylase inhibitor (e.g., valproic acid) as described herein.

As used herein, “non-pluripotent cells” refer to mammalian cells that are not pluripotent cells. Examples of non-pluripotent cells include but are not limited to differentiated cells as well as progenitor cells. Examples of differentiated cells include, but are not limited to, cells from a tissue selected from bone marrow, skin, skeletal muscle, fat tissue and peripheral blood. Exemplary cell types include, but are not limited to, fibroblasts, hepatocytes, myoblasts, neural cells, osteoblasts, osteoclasts, and T-cells.

Cells can be from, e.g., humans or non-human mammals. Exemplary non-human mammals include, but are not limited to, mice, rats, cats, dogs, rabbits, guinea pigs, hamsters, sheep, pigs, horses, and bovines.

Previous studies have recently shown that retrovirus-mediated transfection with four transcription factors (Oct-3/4, Sox2, KLF4 and c-Myc), which are highly expressed in ESCs, into mouse fibroblasts has resulted in generation of induced pluripotent stem (iPS) cells. See, Takahashi, K. & Yamanaka, S. Induction of pluripotent stem cells from mouse embryonic and adult fibroblast cultures by defined factors. Cell 126, 663-676 (2006); Okita, K., Ichisaka, T. & Yamanaka, S. Generation of germline-competent induced pluripotent stem cells. Nature 448, 313-317 (2007); Wernig, M. et al. In vitro reprogramming of fibroblasts into a pluripotent ES-cell-like state. Nature 448, 318-324 (2007); Maherali, N. et al. Directly reprogrammed fibroblasts show global epigenetic remodeling and widespread tissue contribution. Cell Stem Cell 1, 55-70 (2007); Meissner, A., Wernig, M. & Jaenisch, R. Direct reprogramming of genetically unmodified fibroblasts into pluripotent stem cells. Nature Biotechnol. 25, 1177-1181 (2007); Takahashi, K. et al. Induction of pluripotent stem cells from adult human fibroblasts by defined factors. Cell 131, 861-872 (2007); Yu, J. et al. Induced pluripotent stem cell lines derived from human somatic cells. Science 318, 1917-1920 (2007); Nakagawa, M. et al. Generation of induced pluripotent stem cells without Myc from mouse and human fibroblasts Nature Biotechnol. 26, 101-106 (2007); Wernig, M., Meissner, A., Cassady, J. P. & Jaenisch, R.

While it has become accepted that the four transcription factors (Oct-3/4, Sox2, KLF4 and c-Myc) can be used to generate iPSCs, it has also been found that one or more of these transcription factors can be dispensable depending on the conditions and cells used. Recent studies have shown that one of the previously required four genes, cMyc, is dispensable for overexpression in generating iPS cells. See, Nakagawa, M. et al. Generation of induced pluripotent stem cells without Myc from mouse and human fibroblasts Nature Biotechnol. 26, 101-106 (2007); Wernig, M., Meissner, A., Cassady, J. P. & Jaenisch, R. c-Myc is dispensable for direct reprogramming of mouse fibroblasts. Cell Stem Cell 2, 10-12 (2008).

Moreover, small molecules have been found to improve induction of pluripotency and even “replace” one or more of the four transcription factors. See, e.g., Baker, Nature Reports Stem Cells, published online: 13 Nov. 2008; Shi, Y. et al., Cell Stem Cell 3, 568-574 (2008) (e.g., using BIX-01294 and/or the calcium-channel agonist, BayK8644); Kalani, M. Y. S. Proc. Natl. Acad. Sci. USA 105, 16970-16975 (2008); Lluis, F., et al. Cell Stem Cell 3, 493-507 (2008).

Recently, it has been found that it is not necessary to have permanent integration of expression cassettes encoding the transcription factors to generate iPSC cells. For example, transposon technology can be used to extract the expression cassettes. See, Woltjens, et al., Nature, 2009 Apr. 9; 458(7239):766-70; and Yusa, et al., Nature Methods 6(5):363 (2009).

Finally, it has been found that the transcription factor proteins themselves, when fused with polyarginine or other membrane entry sequences or otherwise introduced into a cell can generate iPCS cells. See, e.g., Zhou, Cell Stem Cell (2009).

Any of the above methods, alone or in combination can be used to generate iPSCs that are then screened by the above method.

In some embodiments, transcription factor (i.e., iPSC transcription factors) expression is controlled by an inducible promoter. In some embodiments, the inducible promoter can allow for higher and/or more prolonged expression of iPSC transcription factors compared to non-inducible promoters in the same expression construct (e.g., in some cases, the non-inducible promoter is silenced in the cell). The inventors have found that use of the tetracycline inducible operator tetO to control transcription factor (e.g., at least or more of Oct-3/4, Sox2, KLF4 and c-Myc) expression, in conjunction with a reverse tet transactivator, is particularly useful for generation of iPSCs capable of generating an adult animal in a tetraploid complementation assay.

In these embodiments, the tetO regulatory sequence, generally with at least a minimal promoter, is included upstream from polynucleotide sequences encoding one or more of the relevant iPSC transcription factors. In some embodiments, a different expression cassette is used for each transcription factor, each under the control of the tetO regulator. In other embodiments, polycistronic expression cassettes are used in which two or more transcription factor coding sequences are linked by the appropriate sequences such that fewer expression cassettes are required to generate the relevant transcription factor proteins. Following introduction of the expression cassettes under the control of tetO, expression can be induced in the cells with tetracycline, doxycycline, or another tetracycline analog, and the cells can be cultured and selected for iPSCs.

In addition to the expression cassette(s) under the control of tetO, a further expression cassette comprising a promoter operably linked to polynucleotide encoding a reverse tet transactivator is included. In some embodiments, the reverse tet transactivator comprises a heterologous transactivation domain. In some embodiments, the heterologous tranactivation domain comprises the fusion of two heterologous mammalian transactivation domains, e.g., such that the presence of the heterologous transaction domain results in an optimized regulation compared to a native transactivation domain. See, e.g., Go and Ho, J. Gene Med. 4:258-270 (2002). In some embodiments, the two mammalian transactivation domains are a NFκB p65 activation domain and an HSF1 activation domain. In some embodiments, the reverse tet transactivator is rtTAM2.2 (SEQ ID NO:1). See, e.g., Go and Ho, J. Gene Med. 4:258-270 (2002).

Any or all of the above-described expression cassettes can be delivered by vectors known in the art, including but not limited to retroviral (e.g., lentiviral), adenoviral, AAV vector, or other vectors (see further discussion below). It can be particularly desirable to use a high copy number vector for expression of the reverse tet transactivator to ensure tight regulation of the relevant iPSC transcription factors.

Without intending to limit the scope of the present invention, one interpretation of the data presented herein is that to generate iPSCs capable of generating whole animals in a tetraploid complementation assay, it is advantageous to introduce a low (e.g., 1, 2 or 3) number of copies of expression cassettes encoding transcription factors sufficient to induce pluripotency. In some embodiments, for example, where one of the transcription factors is a Myc polypeptide, the expression cassette encoding the Myc polypeptide has only one, or optionally 2-3 copies inserted in the genome.

Moreover, low copy number of insertions of the above-described transcription factor expression cassettes can be optimally combined with high expression (and optionally high copy number of the corresponding expression cassette) of a transactivating protein that activates transcription of the transcription factor expression cassettes. For example, in some embodiments, a transactivating protein is used and expressed such that the transactivating protein results in more expression from the transcription factor expression cassette(s) than would occur under control of the rtTAM2 transactivating protein as described in Urlinger et al., Proc. Natl. Acad. Sci. USA 97:7963-7968 (2000). As shown herein, for example, use of the rtTAM2.2 transactivating protein, which is a stronger transactivator than rtTAM2, is sufficient to induce iPSCs capable of regeneration of whole animals in a tetraploid complementation assay. Those of skill in the art will appreciate that other transactivating systems, aside from rtTAM2.2 can be used to achieve higher levels than are achieved by rtTAM2.

Further, the duration and timing of iPSC induction can be used to optimize efficiency of the methods of the invention. For example, in some embodiments, induction of iPSCs will last at least 13, 14, 15, 16, 17, 18, 19 days, e.g., between 13-30, 15-30, 17-30, 19-30, 13-50, 15-50, 17-50, or 19-50 days. The period of induction refers to the period from (1) initial expression of the iPSC transcription factors, exposure to such transcription factor proteins, and/or small molecules that “replace” such transcription factors, to (2) the time the iPSCs are selected (e.g., developed into individual cell lines and expanded). Thus, for example, when an inducible expression system is used (e.g., DOX-inducible as described herein) it is the period of DOX induction until iPSCs are selected.

In some embodiments, the induction conditions include contacting the cells with a histone deacetylase inhibitor or other compound that alters epigenetic marks. Multiple methods of generating iPSCs resulted in iPS cell lines that could not support the generation of live mice derived entirely from iPSCs. Comparisons of ESCs or iPSCs that generate mice, with iPSCs that failed to generate mice have identified differences in the activity and genomic imprinting of a locus called Gtl2/Dlk1 (Gtl2 locus) which is found on Chromosome 12 in mouse and on Chromosome 14 in humans. See, Stadfeld, et al., Nature. 465(7295):175-81 (2010); Liu et al., J Biol Chem. 285(25):19483-90 (2010). Normal ESCs express genes from one of the two parental chromosomes. Previous non-fully pluripotent IPSC lines have reduced expression from both parental Gtl loci by allowing extra epigenetic marking. These iPSC lines have methylated both Gtl2 loci and also may have distinct histone acetylation and methylation that turns off gene expression which correlates with or causes failure to generate live animals. The methods described herein produce a high frequency of cell lines that generate mice and these lines have an active and properly imprinted (i.e., hemi-methylated and histone acetylated) Gtl2 locus. When the epigenetic remodeling compound, VPA is removed from the method but no other variables are changed, the Gtl2 locus is no longer active or properly imprinted in the majority of cell lines. Therefore inclusion of VPA, or other histone deacetylase inhibitors or epigenetic remodeling compounds, regulates imprinting of the Gtl2 locus and other genomic regions that control pluripotency. Proper expression of these genes can be observed in cultures without VPA but at a much lower frequency as shown in the table below.

VPA Treatment increases % iPS Cell Lines with Active Gtl2 Locus

| % Locus Unmethylated | VPA− (n = 9) | VPA+ (n = 15) |
|---|---|---|
| High (>40%) | 0% | 27% |
| Medium (10-40%) | 11% | 33% |
| Low (1-10%) | 11% | 40% |
| Negative (not detectable) | 78% | 0% |

Exemplary chromatin modifiers or histone deacetylase inhibitors include, but are not limited to, TSA (trichostatin A) (see, e.g., Adcock, British Journal of Pharmacology 150:829-831 (2007)), VPA (valproic acid) (see, e.g., Munster, et al., Journal of Clinical Oncology 25:18 S (2007): 1065), sodium butyrate (NaBu) (see, e.g., Han, et al., Immunology Letters 108:143-150 (2007)), SAHA (suberoylanilide hydroxamic acid or vorinostat) (see, e.g., Kelly, et al., Nature Clinical Practice Oncology 2:150-157 (2005)), sodium phenylbutyrate (see, e.g., Gore, et al., Cancer Research 66:6361-6369 (2006)), depsipeptide (FR901228, FK228) (see, e.g., Zhu, et al., Current Medicinal Chemistry 3(3):187-199 (2003)), trapoxin (TPX) (see, e.g., Furumai, et al., PNAS 98(1):87-92 (2001)), cyclic hydroxamic acid-containing peptide 1 (CHAP1) (see, Furumai supra), MS-275 (see, e.g., Carninci, et al., WO2008/126932, incorporated herein by reference)), LBH589 (see, e.g., Goh, et al., WO2008/108741 incorporated herein by reference) and PXD101 (see, Goh, supra). In some embodiments, 0.01-100 mM, e.g., 0.1-50 mM, e.g., 1-10 mM of the histone deacetylase inhibitor (including but not limited to those listed above) is used. Note that while induction of iPSCs and contact with the histone deacetylase inhibitor can occur simultaneously (as described in the examples), one can also perform the two steps serially or partially “overlapped.”

### IV. Transformation

This invention relies on routine techniques in the field of recombinant genetics. Basic texts disclosing the general methods of use in this invention include Sambrook et al., Molecular Cloning, A Laboratory Manual (3rd ed. 2001); Kriegler, Gene Transfer and Expression: A Laboratory Manual (1990); and Current Protocols in Molecular Biology (Ausubel et al., eds., 1994)).

In some embodiments, the species of cell and protein to be expressed is the same. For example, if a mouse cell is used, a mouse ortholog is introduced into the cell. If a human cell is used, a human ortholog is introduced into the cell.

It will be appreciated that where two or more proteins are to be expressed in a cell, one or multiple expression cassettes can be used. For example, where one expression cassette is to express multiple polypeptides, a polycistronic expression cassette can be used.

A. Plasmid Vectors

In certain embodiments, a plasmid vector is contemplated for use to transform a host cell. In general, plasmid vectors containing replicon and control sequences which are derived from species compatible with the host cell are used in connection with these hosts. The vector can carry a replication site, as well as marking sequences which are capable of providing phenotypic selection in transformed cells.

B. Viral Vectors

The ability of certain viruses to infect cells or enter cells via receptor-mediated endocytosis, and to integrate into host cell genome and express viral genes stably and efficiently have made them attractive candidates for the transfer of foreign nucleic acids into cells (e.g., mammalian cells). Non-limiting examples of virus vectors that may be used to deliver a nucleic acid of the present invention are described below.

i. Adenoviral Vectors

A particular method for delivery of the nucleic acid involves the use of an adenovirus expression vector. Although adenovirus vectors are known to have a low capacity for integration into genomic DNA, this feature is counterbalanced by the high efficiency of gene transfer afforded by these vectors. “Adenovirus expression vector” is meant to include those constructs containing adenovirus sequences sufficient to (a) support packaging of the construct and (b) to ultimately express a tissue or cell-specific construct that has been cloned therein. Knowledge of the genetic organization or adenovirus, a ˜36 kb, linear, double-stranded DNA virus, allows substitution of large pieces of adenoviral DNA with foreign sequences up to 7 kb (Grunhaus et al., Seminar in Virology, 200(2):535-546, 1992)).

ii. AAV Vectors

The nucleic acid may be introduced into the cell using adenovirus assisted transfection. Increased transfection efficiencies have been reported in cell systems using adenovirus coupled systems (Kelleher and Vos, Biotechniques, 17(6):1110-7, 1994; Cotten et al., Proc Natl Acad Sci USA, 89(13):6094-6098, 1992; Curiel, Nat Immun, 13(2-3):141-64, 1994.). Adeno-associated virus (AAV) is an attractive vector system as it has a high frequency of integration and it can infect non-dividing cells, thus making it useful for delivery of genes into mammalian cells, for example, in tissue culture (Muzyczka, Curr Top Microbiol Immunol, 158:97-129, 1992) or in vivo. Details concerning the generation and use of rAAV vectors are described in U.S. Pat. Nos. 5,139,941 and 4,797,368, each incorporated herein by reference.

iii. Retroviral Vectors

Retroviruses have promise as gene delivery vectors due to their ability to integrate their genes into the host genome, transferring a large amount of foreign genetic material, infecting a broad spectrum of species and cell types and of being packaged in special cell-lines (Miller et al., Am. J. Clin. Oncol., 15(3):216-221, 1992).

In order to construct a retroviral vector, a nucleic acid (e.g., one encoding gene of interest) is inserted into the viral genome in the place of certain viral sequences to produce a virus that is replication-defective. To produce virions, a packaging cell line containing the gag, pol, and env genes but without the LTR and packaging components is constructed (Mann et al., Cell, 33:153-159, 1983). When a recombinant plasmid containing a cDNA, together with the retroviral LTR and packaging sequences is introduced into a special cell line (e.g., by calcium phosphate precipitation for example), the packaging sequence allows the RNA transcript of the recombinant plasmid to be packaged into viral particles, which are then secreted into the culture media (Nicolas and Rubinstein, In: Vectors: A survey of molecular cloning vectors and their uses, Rodriguez and Denhardt, eds., Stoneham: Butterworth, pp. 494-513, 1988; Temin, In: Gene Transfer, Kucherlapati (ed.), New York: Plenum Press, pp. 149-188, 1986; Mann et al., Cell, 33:153-159, 1983). The media containing the recombinant retroviruses is then collected, optionally concentrated, and used for gene transfer. Retroviral vectors are able to infect a broad variety of cell types. However, integration and stable expression typically involves the division of host cells (Paskind et al., Virology, 67:242-248, 1975).

Lentiviruses are complex retroviruses, which, in addition to the common retroviral genes gag, pol, and env, contain other genes with regulatory or structural function. Lentiviral vectors are well known in the art (see, for example, Naldini et al., Science, 272(5259):263-267, 1996; Zufferey et al., Nat Biotechnol, 15(9):871-875, 1997; Blomer et al., J Virol., 71(9):6641-6649, 1997; U.S. Pat. Nos. 6,013,516 and 5,994,136). Some examples of lentivirus include the Human Immunodeficiency Viruses: HIV-1, HIV-2 and the Simian Immunodeficiency Virus: SIV. Lentiviral vectors have been generated by multiply attenuating the HIV virulence genes, for example, the genes env, vif, vpr, vpu and nef are deleted making the vector biologically safe.

Recombinant lentiviral vectors are capable of infecting non-dividing cells and can be used for both in vivo and ex vivo gene transfer and expression of nucleic acid sequences. For example, recombinant lentivirus capable of infecting a non-dividing cell wherein a suitable host cell is transfected with two or more vectors carrying the packaging functions, namely gag, pol and env, as well as rev and tat is described in U.S. Pat. No. 5,994,136, incorporated herein by reference. One may target the recombinant virus by linkage of the envelope protein with an antibody or a particular ligand for targeting to a receptor of a particular cell-type. By inserting a sequence (including a regulatory region) of interest into the viral vector, along with another gene which encodes the ligand for a receptor on a specific target cell, for example, the vector is now target-specific.

C. Vector Delivery and Cell Transformation

Suitable methods for nucleic acid delivery for transformation of a cell, a tissue or an organism for use with the current invention are believed to include virtually any method by which a nucleic acid (e.g., DNA) can be introduced into a cell, a tissue or an organism, as described herein or as would be known to one of ordinary skill in the art. Such methods include, but are not limited to, direct delivery of DNA such as by ex vivo transfection (Wilson et al., Science, 244:1344-1346, 1989, Nabel and Baltimore, Nature 326:711-713, 1987), optionally with Fugene6 (Roche) or Lipofectamine (Invitrogen), by injection (U.S. Pat. Nos. 5,994,624, 5,981,274, 5,945,100, 5,780,448, 5,736,524, 5,702,932, 5,656,610, 5,589,466 and 5,580,859, each incorporated herein by reference), including microinjection (Harland and Weintraub, J. Cell Biol., 101:1094-1099, 1985; U.S. Pat. No. 5,789,215, incorporated herein by reference); by electroporation (U.S. Pat. No. 5,384,253, incorporated herein by reference; Tur-Kaspa et al., Mol. Cell Biol., 6:716-718, 1986; Potter et al., Proc. Nat'l Acad. Sci. USA, 81:7161-7165, 1984); by calcium phosphate precipitation (Graham and Van Der Eb, Virology, 52:456-467, 1973; Chen and Okayama, Mol. Cell Biol., 7(8):2745-2752, 1987; Rippe et al., Mol. Cell Biol., 10:689-695, 1990); by using DEAE-dextran followed by polyethylene glycol (Gopal, Mol. Cell Biol., 5:1188-1190, 1985); by direct sonic loading (Fechheimer et al., Proc. Nat'l Acad. Sci. USA, 84:8463-8467, 1987); by liposome mediated transfection (Nicolau and Sene, Biochim. Biophys. Acta, 721:185-190, 1982; Fraley et al., Proc. Nat'l Acad. Sci. USA, 76:3348-3352, 1979; Nicolau et al., Methods Enzymol., 149:157-176, 1987; Wong et al., Gene, 10:87-94, 1980; Kaneda et al., Science, 243:375-378, 1989; Kato et al., J Biol. Chem., 266:3361-3364, 1991) and receptor-mediated transfection (Wu and Wu, Biochemistry, 27:887-892, 1988; Wu and Wu, J. Biol. Chem., 262:4429-4432, 1987); each incorporated herein by reference); and any combination of such methods.

### V. Kits

The present invention also provides kits, e.g., for use in inducing or improving efficiency of induction of pluripotency in cells. Such kits can comprise any or all of the reagents described herein, including but not limited to:

expression cassettes comprising one or more transcription factor expression cassette(s) into non-pluripotent animal cells, which expression cassette(s) comprise a promoter operably linked to a polynucleotide encoding one or more transcription factors sufficient to induce pluripotency into the cells, where expression of the transcription factors is controlled by a tetracycline and/or doxycycline-inducible tetO regulatory element; and/or
a transcriptional activator expression cassette comprising a promoter operably linked to a polynucleotide encoding a tetracycline and/or doxycycline responsive transcriptional activator, the transcriptional activator comprising a reverse tet repressor fused to a heterologous transactivation domain.

In some embodiments, the heterologous transactivation domain comprises the fusion of two heterologous mammalian transactivation domains. In some embodiments, the two mammalian transactivation domains are a NFκB p65 activation domain and an HSF1 activation domain. In some embodiments, the transactivation domain is rtTAM2.2 (e.g., as described in Go and Ho, J. Gene Med. 4:258-270 (2002)).

Alternatively, or in combination with one or more of the above-described expression cassettes, the kits can also comprise, e.g., a recombinant expression cassette comprising an adult-specific promoter operably linked to a polynucleotide encoding a recombinase; and a recombinase site expression cassette comprising: (1) a promoter operably linked to a first reporter polynucleotide; and (2) a second reporter polynucleotide, wherein the first reporter polynucleotide is spanned by recombinase sites such that the promoter controls expression of the first reporter polynucleotide prior to contact of the recombinase to the recombinase site expression cassette and such that the promoter controls expression of the second reporter polynucleotide upon contact of the recombinase-initiated recombination of the recombinase site expression cassette.

In any of the embodiments described herein, the kit can also include one or more histone deacetylase inhibitors or chromatin modifiers (e.g., VPA).

### Examples

The following examples are offered to illustrate, but not to limit the claimed invention.

Recent landmark experiments have shown that transient overexpression of a small number of transcription factors can reprogram differentiated cells into induced pluripotent stem cells (iPSCs) that resemble embryonic stem cells (ESCs)¹⁻⁷. These iPSCs hold great promise for medicine because they have the potential to generate patient-specific cell types for cell replacement therapy and produce in vitro models of disease, without requiring embryonic tissues or oocytes.⁸⁻¹⁰. While current iPSC lines resemble ESCs, they have not passed the most stringent test of pluripotency by generating full-term or adult mice in tetraploid complementation assays^{3, 11}, raising questions as to whether they are sufficiently potent to generate all the cell types in an organism. Whether this difference between iPSCs and ESCs reflects intrinsic limitations of direct reprogramming is not known. Here, we report fertile adult mice derived entirely from iPSCs that we generated by inducible genetic reprogramming of mouse embryonic fibroblasts (MEFs). Producing adult mice derived entirely from a reprogrammed fibroblast shows that all features of a differentiated cell can be restored to an embryonic level of pluripotency without exposure to unknown ooplasmic factors. Comparing these fully pluripotent iPSC lines to less developmentally potent lines may reveal molecular markers of different pluripotent states. In addition, mice derived entirely from iPSCs will provide a novel resource to assess the functional and genomic stability of cells and tissues derived from iPSCs, which is important to validate their utility in cell replacement therapy and research applications.

Historically, the only way to generate an adult mammal was by fertilization. Advances in somatic cell nuclear transfer (SCNT) have now produced genetically identical mouse clones from a variety of differentiated cell types, from fibroblasts to neurons^{12, 13}. Similarly, genetically identical adult mice may be derived from ESCs (or SCNT-ESCs) by tetraploid blastocyst complementation, in which all adult tissues derive from the stem cell line while extraembryonic tissues are supplied by the tetraploid cells.^{14, 15}. For unknown reasons, current iPSC lines have not generated adult or full term mice in tetraploid complementation assays. This finding, and recent reports of reproducible gene expression differences between iPSCs and ESCs, suggests that direct reprogramming may be insufficient to restore differentiated cells to full pluripotency, as measured by ESC equivalence^{3, 11, 16}. Autonomous generation of mice from iPSCs would validate direct reprogramming as equivalent to reprogramming by SCNT, establish iPSCs as appropriate functional substitutes for ESCs and provide a new method to generate adult mice from differentiated cells.

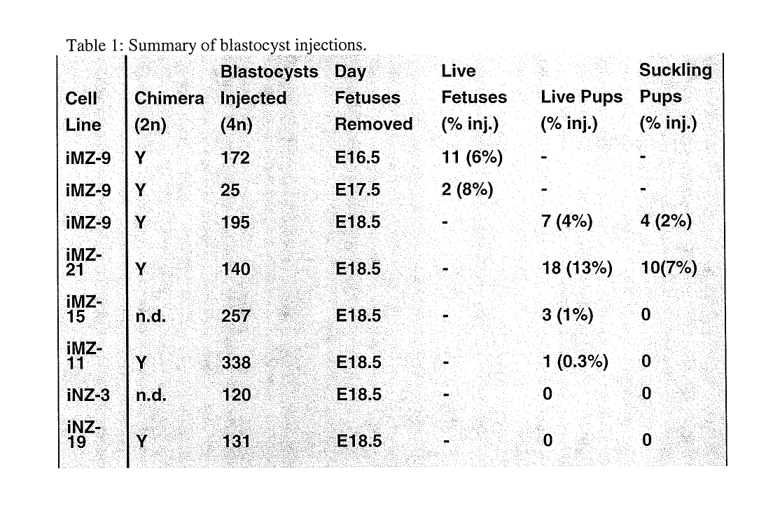

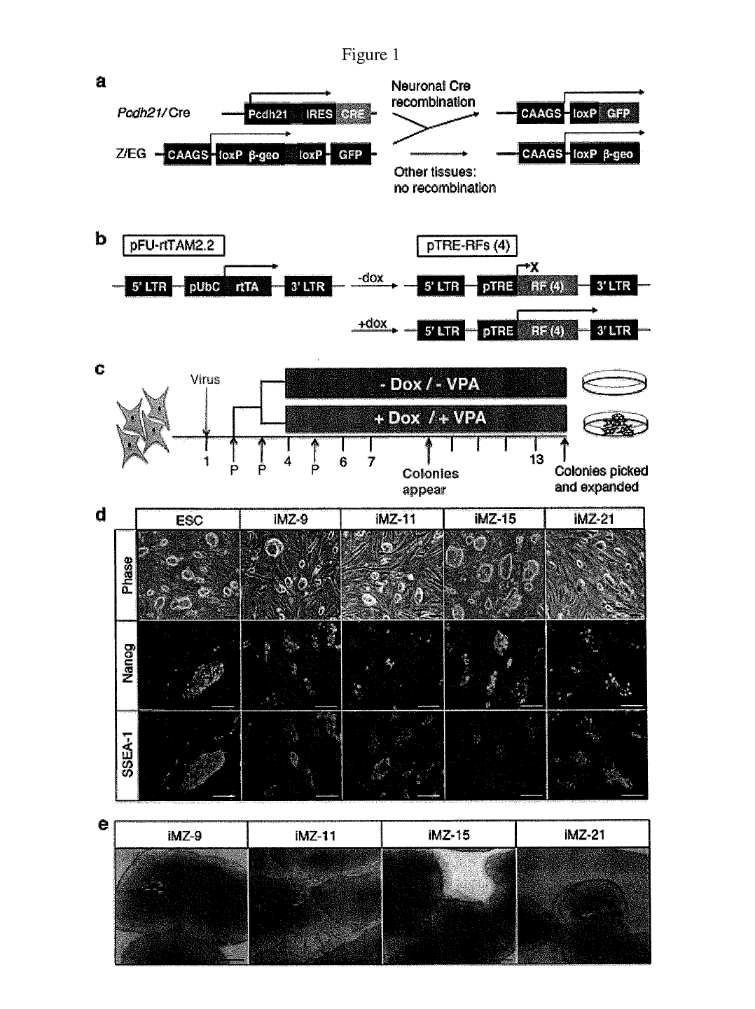

To conclusively demonstrate that iPSC lines can generate adult mice in tetraploid complementation assays, we designed a genetic marking strategy to distinguish between host blastocyst and iPS-derived cells. We established mouse embryonic fibroblasts (MEFs) from animals generated by a cross of two mouse lines (Pcdh21/Cre and Z/EG, FIG. 1a). The Z/EG transgene labels the majority of cells in an animal with a visible marker (β-geo, a fusion of the β-galactosidase and neomycin genes)¹⁷ while the Pcdh21/Cre modification results in Cre expression in rare neuronal subtypes, but not in ESCs¹⁸. Cre expression causes excision of the floxed β-geo gene, resulting in GFP expression in olfactory bulb mitral cells, a feature we exploit later (FIG. 1a).

We reasoned that inappropriate expression of reprogramming genes during development could inhibit embryonic and postnatal development. Therefore, we designed a drug-inducible lentiviral reprogramming strategy to achieve tight control of transgene expression in iPSCs and their derivatives (FIG. 1b)¹⁹. The four original reprogramming factors (Oct4, Sox2, Klf4 and cMyc) were placed under control of the tetO promoter, which is activated by the reverse tetracycline transactivator (rtTA) protein in the presence of the tetracycline analog doxycycline (dox). We used an enhanced version of the rtTA transcriptional activator protein (rtTAM2.2) that induces higher gene expression levels than the rtTAM2 protein²⁰. To promote complete reprogramming and facilitate isolation of fully reprogrammed iPSCs we exposed MEFs to the histone deacetylase inhibitor valproic acid (VPA), which has been reported to enhance reprogramming efficiency²¹ and to select against incompletely reprogrammed cells by inhibiting cell division²² (Supplementary methods).

Reprogramming of Pcdh21/Cre-Z/EG fibroblasts by this method resulted in colonies after five (dox+,VPA+) to seven (dox only) days of dox induction (FIG. 1c, d, Supplementary methods). No colonies emerged in the absence of dox, which demonstrates both the inducibility and specificity of our system.

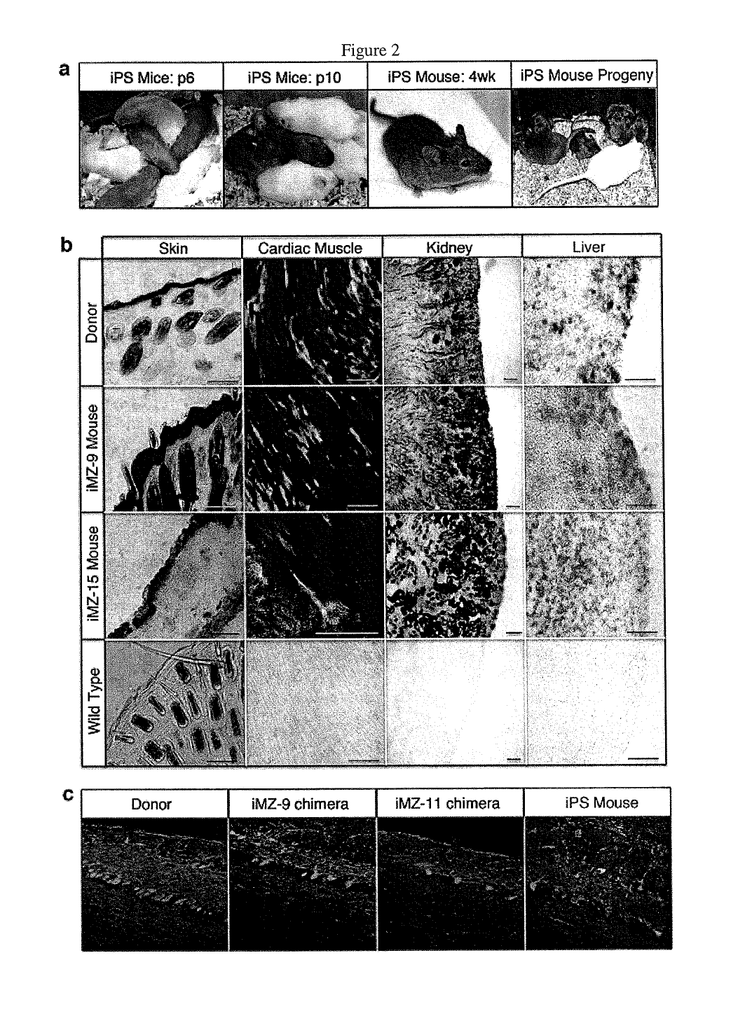

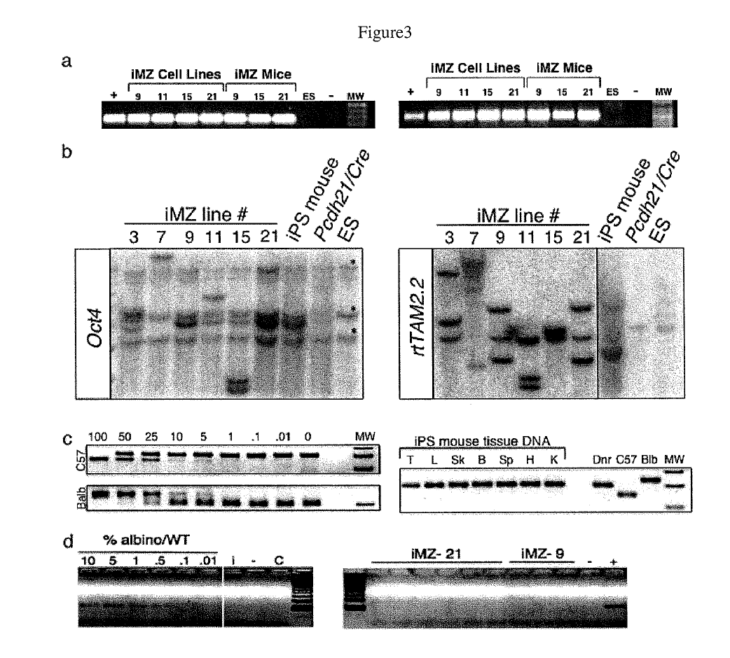

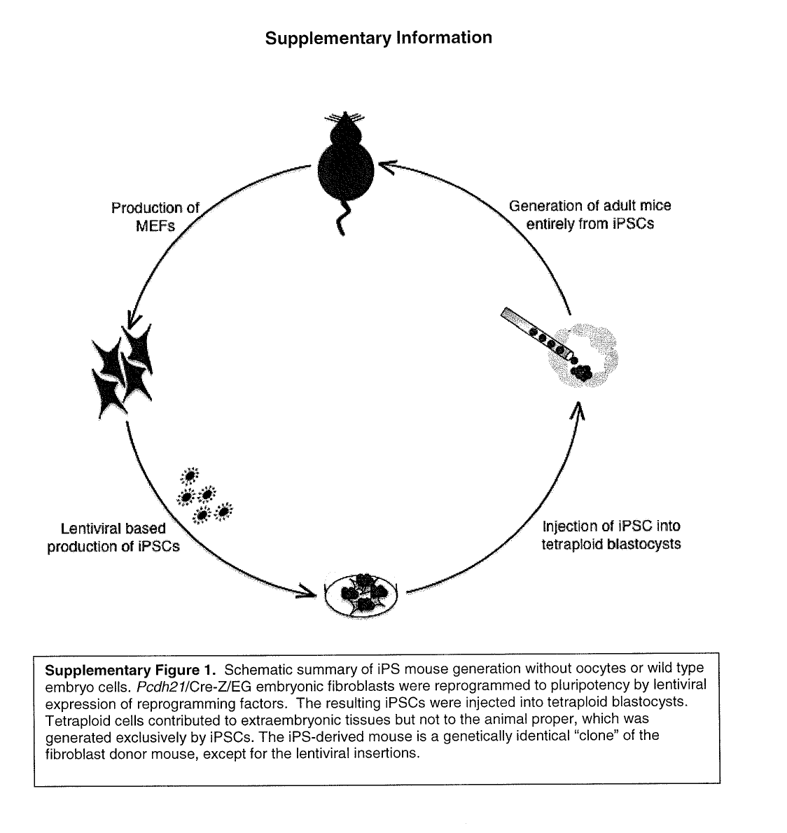

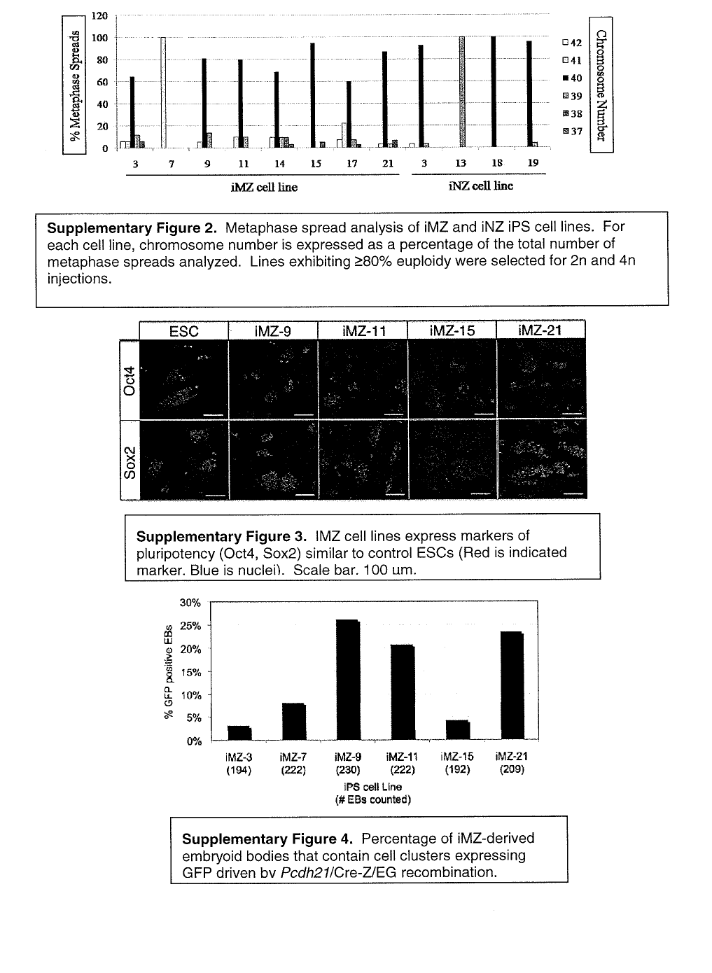

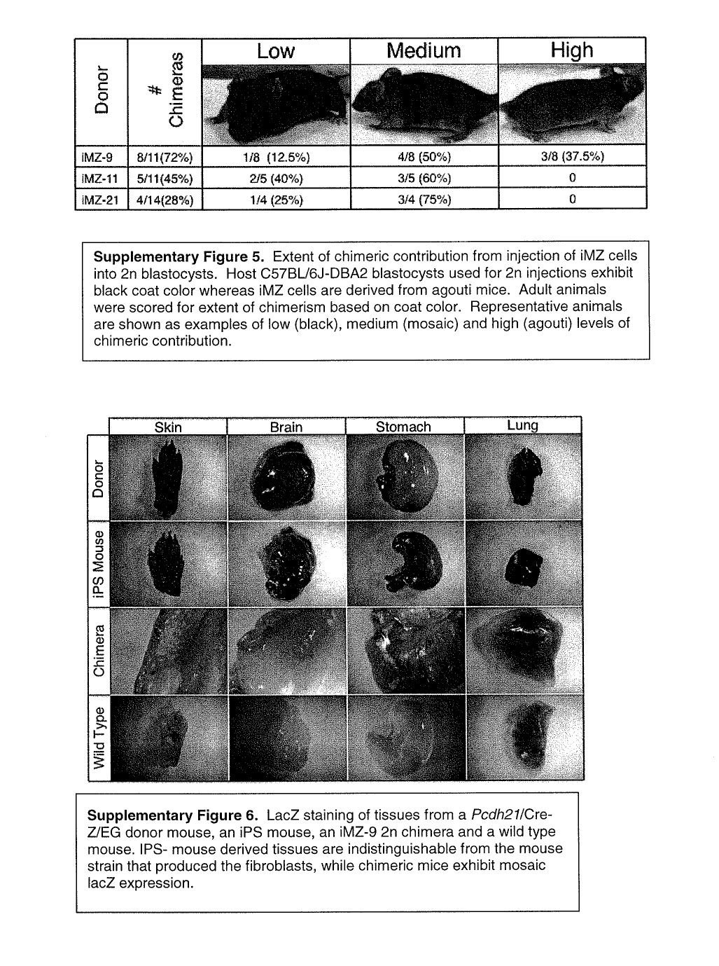

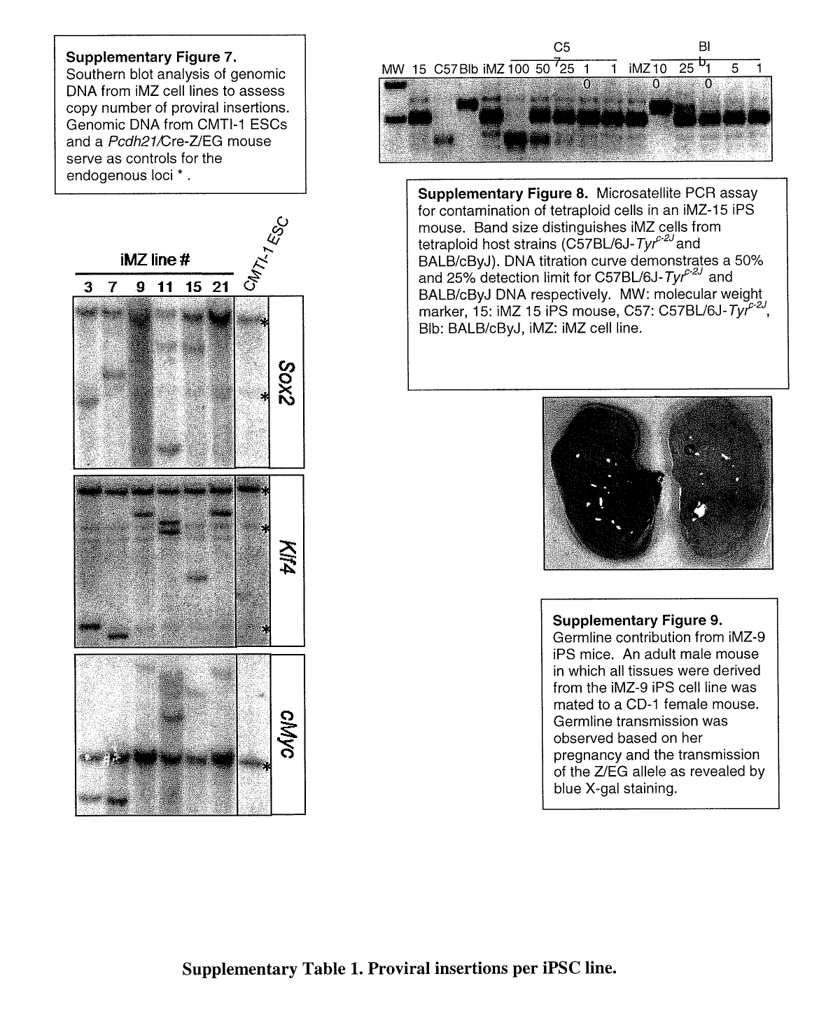

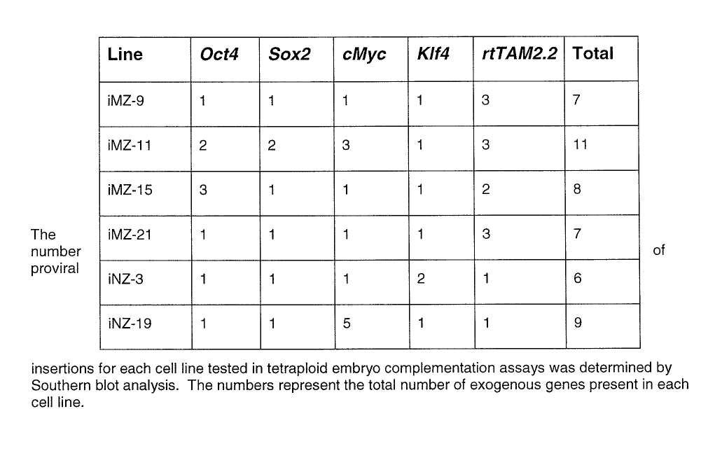

At present there is no established method to select iPSCs that will contribute to all tissues of an organism. To prioritize the iMZ lines for tetraploid complementation assays, we assessed lines for similarity to ESC lines by morphology, proliferation rate, expression of pluripotency markers and ability to generate embryoid bodies (EBs) (FIG. 1d, 2d, Supplementary FIGS. 2, 3). We also exploited our cell-type specific Cre line to ask whether EBs made from our iMZ lines could generate cells that resembled olfactory bulb mitral cells based on neuronal morphology and GFP expression (FIG. 1e, Supplementary FIG. 3). Using these criteria, we selected 12 candidate lines for chromosomal analysis (Supplementary FIG. 4, 5 and Supplementary Methods). To establish the pluripotency of lines generated in this paradigm, we tested three euploid lines (iMZ-9, 11 and 21) in diploid blastocyst injection assays. All tested lines contributed to chimeric mice based on coat color. Line iMZ-9 iPSCs generated multiple mice with nearly 100% agouti fur (Supplementary FIG. 6). These iPSCs contributed to all germ layers based on expression of the β-geo transgene in multiple tissues (Supplementary FIG. 7), production of GFP+ cells in the olfactory bulb (FIG. 2c) and germline transmission of lines iMZ-9 and 11 (data not shown).

Southern blot analyses revealed that lines iMZ-9 and iMZ-21 have identical patterns of proviral insertions and thus, these lines likely derived from the same initial transduced MEF (FIG. 3b, Supplementary FIG. 9). After induction of transgene expression in iPSC derivation, iMZ MEFs were split only once. Therefore, these two independently isolated iPSC lines potentially experienced different stochastic events during reprogramming, which can confer different epigenetic alterations and variable developmental potential upon otherwise identical cell lines^{23, 24}. For these reasons, we refer to iMZ-9 and iMZ-21 as independent lines.

To determine whether these iMZ lines could generate full-term or adult mice, we performed tetraploid complementation assays. In a series of independent experiments, we injected albino tetraploid blastocysts with cells derived from iMZ-9, iMZ-11, iMZ-15, iMZ-21 and two additional iPSC lines (iNZ-3, iNZ-19) (Table 1, Supplementary Methods). We performed Caesarean section on the evening before scheduled delivery and obtained breathing pups, termed iPS mice, with normal morphology from lines iMZ-9 (4 viable pups, 3 either non-viable or cannibalized after fostering, 13 apparently viable on E.16.5 or E17.5), iMZ-21 (10 viable pups, 8 non-viable), iMZ-15 (1 live pup with a herniated umbilical cord, 1 full-term pup with respiratory failure, 1 cannibalized) and iMZ-11 (one live pup, later cannibalized). Lines iNZ-3 or iNZ-19 did not generate full term pups (Table 1, FIG. 2a).

The majority of surviving pups exhibit no obvious morphological abnormalities (FIG. 2a). Non-viable pups typically presented with difficulty breathing, which is common in tetraploid complementation experiments performed with ESCs¹⁴. In each successful experiment, the efficiency of generating iPS mice ranged from 0.3% to 13%, which is similar to published efficiencies for ES and SCNT-ES cells (Table 1)^{15, 25, 26}. Importantly, iMZ-9 iPS cells reproducibly generated adult mice in multiple experiments.

Although tetraploid cells rarely contribute to the embryo proper beyond mid-gestation^{25, 27}, to conclusively demonstrate that these iPS mice derived entirely from the iMZ cell lines we analyzed coat and eye color and performed histological staining. The coat and eye color of the iPS mice (agouti, pigmented) differs from both the albino tetraploid host blastocyst and the albino recipient female. As expected, all pups exhibited pigmented eyes and uniformly agouti fur (FIG. 2a), in contrast to the variation in coat color observed in the diploid chimera assays (Supplementary FIG. 6). Similarly, cells derived from iMZ lines stain positive for β-galactosidase, while blastocyst-derived cells do not. Intact tissues and tissue sections representing all three germ layers displayed positive staining for β-galactosidase (β-gal)(FIG. 2b and Supplementary FIG. 7). We observed no histological differences between iPS mice and the Pcdh21/Cre-Z/EG mouse strain, while staining was clearly different from iMZ chimeric animals (FIG. 2b, c, Supplementary FIG. 7.). In addition, immunofluorescence analyses of the olfactory bulbs of iPS mice revealed that all cells express n-gal (except for the mitral cells which express GFP, as expected) (FIG. 2c).

To exclude minor contributions of tetraploid host blastocyst cells during iPS mouse development, we designed sensitive PCR assays to detect microsatellite markers and albino mutations of the Tyr gene that differ between host blastocysts and iMZ cells (FIG. 3c, d). Analyses of multiple tissues from an iMZ-9 mouse revealed no contribution from host blastocyst cells in the microsatellite assay (FIG. 3c). Similar results were observed with tail tissue from the iMZ-15 mouse (Supplemental FIG. 8). We also performed tests for the albino mutation on DNA from nine individual mice derived from lines iMZ-9 and 21 and detected no contribution of host blastocyst cells (FIG. 3d). These data strongly support the conclusion that the iMZ lines are capable of generating all cell types of adult mice in tetraploid complementation assays, at least to the level that is typical of ESCs.

Importantly, genetic analyses rule out contamination of our iPSC lines by pre-existing ESCs. PCR experiments demonstrated that all iMZ lines and iPS mice carry the Pcdh21/Cre and Z/EG genetic modifications, which do not co-exist in any known ESC lines (FIG. 3a.). Similarly, Southern blot analyses with probes for Oct4, and rtTA-M2.2 show that the patterns of proviral integration of the iPS mice are identical to the patterns of the iPSC lines from which they derived. Furthermore, the relative intensity of the Oct4 proviral insertions and endogenous Oct4 bands is similar as would be expected if the iPS mice derived predominantly from iPSCs (FIG. 3b).

A final test of pluripotency is to establish germline contribution and production of viable offspring. In crosses with albino female mice, male iPS mice derived from line iMZ-9 exhibit germline transmission as evidenced by production of 100% agouti pups and expression of the Z/EG allele in the expected number of progeny (FIG. 2a, Supplementary FIG. 8). Taken together, these data demonstrate that direct reprogramming of MEFs with four factors can generate iPSC lines that possess full pluripotency as measured by production of fertile adult mice in tetraploid complementation assays.

There are several features of our experiments that could be responsible for the enhanced pluripotency of our iPSC lines. First, the high levels of transgene induction afforded by rtTA-M2.2 and the extended duration of reprogramming factor expression in our protocol may more completely reprogram cells. Second, transgene expression in our iPSC lines is regulated by a dox inducible promoter, which may help to prevent inappropriate expression of reprogramming factors during embryonic development. In support of this idea, quantitative RT-PCR experiments demonstrate that proviral transgenes are nearly completely silent in iPSCs the absence of dox (Supplementary FIG. 11). Furthermore, the lines that generate iPS mice most robustly (iMZ9 and 21) have reduced expression of all four reprogramming factors, while less efficient lines (iMZ 11 and 15) have detectable expression of Klf4 and or Oct4 in the absence of dox. Third, previously reported reprogramming experiments either did not use VPA, or used short VPA treatments and did not report tetraploid complementation experiments. Prolonged VPA treatment in our experimental protocol may have allowed resetting of the epigenome to a chromatin state more similar to that of ESCs. The timing and extent of passaging, the genetic background of reprogrammed MEFs and our iPSC selection criteria may have contributed to the enhanced pluripotency of our lines.

We cannot exclude the possibility that the enhanced pluripotency of the iPSC lines we report here is a result of reprogramming of a rare cell type or a particular pattern of proviral insertion. However, we have established three cell lines with distinct proviral integration patterns that can generate full term mice, suggesting that multiple individual MEF cells can lead to fully pluripotent iPCSs and that a particular insertion pattern is not required. At present, the two iMZ lines that generate adult mice derive from a single infected cell. Until additional adult mice are generated we cannot exclude models requiring a rare cell type or particular proviral insertion.

The generation of fertile adult mice from iPSCs serves as an important proof-of-principle validation that reprogramming technology can produce iPSCs with functional equivalence to ESCs. These data demonstrate that direct reprogramming with four factors can recapitulate the reprogramming capacity of the oocyte and show that non-genomic components of differentiated cells, such as mitochondria, do not impede reprogramming to full pluripotency. We speculate that comparing iPSC lines which generate iPS mice with those that cannot generate mice but satisfy other criteria of pluripotency (i.e. chimerism and germline contribution) may reveal important molecular differences associated with states of pluripotency.

### Methods Summary:

iPSCs were derived from E13.5 mouse embryonic fibroblasts using dox-inducible lentiviruses encoding Oct4, Sox2, Klf4 and cMyc as previously described {Wernig, 2008 #23}, except that we used rtTAM2.2 and included VPA treatment. Reprogrammed lines were characterized by immunofluorescence (SSEA-1, Nanog, Oct4, Sox2). In vitro differentiation into embryoid bodies by aggregation in suspension and treatment with all-trans retinoic acid was used to assess the ability of iMZ lines to generate rare GFP+ cells. Karyotype was examined by analysis of metaphase spreads prepared by the hanging drop method. Chimeric mice were produced by injection of euploid iPSC lines into diploid blastocysts {Nagy, 2003 #73}. iPS mice were produced by injection of iPSCs into tetraploid blastocysts generated by electrofusion of two-cell embryos according to established methods {Eggan, 2006 #74} {Nagy, 2003 #73}. Intact tissues and tissue sections from chimeras or iPS mice were stained with X-gal or antibodies for β-galactosidase and GFP. Genetic analyses of iPS mice were performed by Southern blotting for proviral insertions, genotype analysis for the Pcdh21/Cre and Z/EG alleles, and PCR for microsatellite markers and the Tyr^{c-2J} albino mutation using standard methods. Residual expression of virally encoded transgenes was examined by RT-qPCR using lentiviral-specific primers.

Generation of Pcdh21/Cre-Z/EG and Nex/Cre-Z/EG mice. To generate the Pcdh21-Cre mice, an IRES-Cre recombinase-FRT-Neo-FRT cassette was inserted into the Pcdh21 locus immediately following the translational stop sequence by homologous recombination in ESCs. ESC colonies were screened and confirmed by Southern blot. Positive colonies were used to generate chimeric mice and these mice or their Pcdh21/Cre positive offspring were crossed to Z/EG mouse lines to generate the Pcdh21/Cre-Z/EG mouse strain. No ESCs containing both modifications have been produced. Mice retain the FRT-Neo-FRT cassette. Mouse genotypes were confirmed by PCR for the wild-type Pcdh21 allele, the Pcdh21-Cre knock-in allele, and β-geo. Primer sequences and PCR conditions are available upon request. The NEX-Cre mouse line labels post-mitotic neurons in various brain regions^{28, 29}. We crossed this line to the Z/EG line to produce NEX/Cre-Z/EG mice from which the control iNZ fibroblasts were derived.

Generation of Lentiviral Constructs. All lentiviral shuttle vectors were generated from a modified version of the FUGW vector^{30, 31}. To generate pFU-rtTA, the rtTAM2.2 gene³² was cloned from the pWG020 vector into the XbaI and BamHI sites of the viral vector MCS. A linker containing an additional BamHI site, a kozak sequence and a start codon was inserted into the XbaI site. The doxycycline (dox) inducible lentiviral construct, pFT-MCS, was generated by replacing the human ubiquitin C promoter with seven tetO repeats followed by a minimal CMV promoter. The dox inducible promoter was amplified from pTRE-d2eGFP (BD Biosciences, Clontech) and cloned into the PacI and XbaI sites of the FUGW derived vector. The coding sequences of Oct4, Sox2, cMyc, and Klf4 were ligated into pFT-MCS. Oct4, Sox2, and Klf4 were inserted into the EcoRI site. cMyc was inserted using the XbaI and BamHI sites.

Production of lentivirus. Virus was produced in HEK293T cells by calcium phosphate co-transfection of lentiviral shuttle vectors with the pCMVΔ8.9 and pVSVg viral packaging vectors. Virus was harvested at 24, 48, and 72 hs post-transfection and concentrated by ultracentrifugation (2 hs at 25,000 rpm at 4° C.).

Generation of iPSCs. Mouse embryonic fibroblasts (MEFs) were prepared from Pcdh21/Cre-Z/EG (iMZ lines) or Nex/Cre-Z/EG (iNZ lines) E13.5 embryos. Generation of iMZ lines: After 24 hours in culture, individual wells of ˜300,000 MEFs were transduced with lentiviruses (day 1) and split 1:2 (day 2) and 1:3 (day 3) to generate 6 wells of transduced MEFs. On post-transduction day 4, dox (10 μg/ml) was added to four wells to induce expression of reprogramming genes; three of these wells were also treated with VPA (1.9 mM). The remaining two wells were treated with nothing, or VPA alone. To maintain conditions for optimal MEF growth and viability, on post-transduction day five, the three dox+VPA wells were expanded into a 15 cm² dish while the other three conditions were expanded to 10 cm² dishes. On post-transduction day 9 (five days after dox induction), colonies were observed in the dox+VPA wells; colonies emerged in the dox only wells on day 11 (seven days after dox induction). No colonies emerged in the absence of dox, with or without VPA. On post-transduction day 12, eight small colonies (iMZ-1-8) were isolated from the dox+VPA plate by aspiration into a p10 pipette tip, brief trypsinization, and plating to mitotically inactive MEF feeders in a 96 well dish. On day 14, additional colonies were isolated, of which 12 were expanded (lines iMZ-9-21) and characterized further. Lines iMZ-1, 4, 5, 6, 8 and 16 did not grow or proliferated more slowly than ESC controls, so these were not maintained. On post-transduction day 17, all putative iPSC lines were transferred to fresh feeders in single 96 well plates. Cell lines were expanded into two wells on post-transduction day 21. Cells were maintained in dox+VPA until post-transduction day 23, when both were removed. All cell lines appeared to maintain ESC like morphology and proliferation rates. Lines were expanded to 24 well plates on day 24. Subsequently, cell lines were banked and maintained as described in the cell culture conditions section. Generation of iNZ lines: Initial reprogramming conditions were identical to those of iMZ lines except that cells were split 1:2 on post-transduction day 3 to generate four wells (dox+VPA, dox only, VPA only and no treatment). Cells were split once after dox and VPA addition. In this experiment, colonies were observed on post-transduction day 11 in wells with dox+VPA and dox only but not in wells lacking dox, as expected. VPA treatment was halted for one day at day 12 to allow proliferation and then resumed while dox treatment was continuous. On day 14, 39 colonies were observed in dox+VPA (20) and dox only (19) wells. This would represent a reprogramming efficiency of 0.02% (20 colonies/75,000 initial fibroblasts), which does not take into account the transduction efficiency for all five viruses, or the expansion of clones with identical insertions. After splitting the dox+VPA wells, 32 colonies from the dox+VPA wells were isolated and 19 maintained proliferation at rates similar to ES cells. These lines are called iNZ-1-19.

Cell Culture Conditions. ESCs and iPSCS were maintained on mitotically inactivated MEF feeders in 85% DMEM, 15% ESC qualified FBS (Gibco), 1 mM L-glutamine, 0.1 mM non-essential amino acids, 0.1 mM 2-mercaptoethanol, 1000 units of ESGRO/ml (Chemicon) 100 units/ml penicillin and 10 μg/ml streptomycin. MEF feeders were maintained on 0.1% gelatin-coated dishes in 70% DMEM, 20% Medium 199, 10% FBS and 100 U/ml penicillin/streptomycin. All cells were kept at 37° C. in a humidified environment at 5% CO₂. Embryoid bodies were aggregated in suspension using ultra-low attachment surfaces (Corning) in ESC medium lacking ESGRO and 2-mercaptoethanol and treated with 2×10⁻⁶ M all-trans retinoic acid (Sigma) from days 4-10.

Southern Blotting. Genomic DNA was prepared using the DNAeasy Blood and Tissue Kit (Qiagen). Eight micrograms of DNA were digested with PvuII (Oct4), BamHI (Sox2, Klf4, cMyc) or EcoRI (rtTAM2.2), resolved on 0.8% agarose gels, transferred to Hybond-N+ membrane (Amersham Biosciences) and hydridized with radiolabeled-probe at 65° C. Probes were generated using the Prime-It II Random Primer Labeling Kit (Stratagene). Images were captured on a Typhoon 8600 Variable Mode Imager and analyzed with ImageQuant 5.2 software. The open reading frame (ORF) of each gene served as template for probe synthesis. Oct4 ORF (NM_{—}013633)=1,058 bp; Sox2 ORF (NM_{—}011443)=959 bp; cMyc ORF (NM_{—}010849)=1,364 bp, Klf4 ORF (NM_{—}010637)=1,451 bp and rtTAm2.2 ORF=1,490 bp. Following hybridization, blots were successively washed with 2×SSC/0.1% SDS at room temperature (RT) and 0.2×SSC/0.1% SDS at 65° C.

Immunofluorescence analyses of cell lines. Cells were fixed with 4% paraformaldehyde (PFA) at RT for 20 min, blocked and permeabilized for 1 h at RT in PBS/Triton-X-100 (0.1%), incubated overnight at 4° C. in primary antibodies against Oct4 (Santa Cruz Biotechnology, 1:100), SSEA1 (Developmental Studies Hybridoma Bank, 1:500), Nanog (Cosmo Bio Co., 1:50), Sox2 (R&D Systems, 1:50), washed in blocking solution 3×15 min, incubated for 30 min at RT with fluorescence conjugated secondary antibodies (Alexa). Nuclei were labeled with DAPI or TOTO-3 (Molecular Probes, Invitrogen). Images were collected on an Olympus BX60M microscope and analyzed with MetaMorph software.

Analyses of tissues and tissue sections. Whole tissues were dissected and placed directly into X-gal staining buffer (100 mM sodium phosphate pH 7.3, 2 mM MgCl₂, 0.01% sodium deoxycholate 0.02% NP-40, 5 mM potassium ferricyanide, 5 mM potassium ferrocyanide, 1 mg/ml X-gal) and incubated at 37° C. until staining of controls was evident (several hours). Tissues were washed in PBS and preserved in 4% PFA/PBS fixative. For sections, tissues were collected and fixed with 4% PFA/PBS for 1 h at 4° C., 30% sucrose protected overnight at 4° C., OCT embedded and cut into 30 μM sections using a Leica CM 3050S Cryostat. Sections were air dried on charged slides for 20 min and fixed in 4% PFA for 7 min. Sections were then X-gal stained for 2-3 h at 37° C., mounted and imaged on an Olympus AX70 microscope and analyzed with Spot imaging software. Alternatively, brain slices were co-stained with primary antibodies against LacZ (Promega, 1:500) and GFP (Invitrogen, 1:500) and imaged on an Olympus Fluoview FV500 LSM microscope. Images were analyzed using MetaMorph software.

Microsatellite PCR assay. The length of the microsatellite detected by the D12Mit136 primer pair is different in each of the Pcdh21/Cre-Z/EG, C57BL/6J-Tyr^{c-2J} and BALB/c ByJ mouse strains. The genotype of the host tetraploid blastocysts varied in experiments but in each case tetraploid blastocysts will carry either the Balb/C allele or both the Balb/C and the C57BL/6J alleles. Expected bands for C57BL/6J, BALB/c, Pcdh21/Cre-Z/EG are 147, 213 and 100 bp, respectively. Primer sequences are: D12Mit136 sense: 5′-TTTAATTTTGAGTGGGTTT GGC-3′; antisense: 5′-TGGCTACATGTACACTGATCTCCA-3′. PCR conditions were 94° C. for 2 min, 43 cycles of 94° C. for 1 min, 53° C. for 15 s, 72° C. for 45 s.

Albino allele PCR assay. Tetraploid blastocysts carry the C57BL/6J-Tyr^{c-2J} albino mutation, while the iMZ and iNZ iPSCs do not. We designed a PCR assay in which the 3′ end of the sense primer is specific for the Tyr^{c-2J} mutation. C57BL/6J-Tyr^{c-2J} DNA yields the expected 115 bp product, whereas no product is observed with iMZ-9 DNA. DNA was harvested from Pcdh21/Cre-Z/EG control and iPS mouse tissue by proteinase K digestion followed by phenol/chloroform extraction and ethanol precipitation. Primers used were: sense 5′-TCAAAGGGG TGGATGACCT-3′ and antisense 5′-CCCCCAAATCCAAACTTACA-3′. PCR conditions were 94° C. for 2 min, 40 cycles of 94° C. for 1 min 65° C. for 15 s, 72° C. for 20 s).

RT-qPCR. RT-qPCR. RNA was harvested from iPSC lines maintained in the absence of dox (−dox) or treated with 10 μg/ml dox for 24 hs to re-induce proviral encoded transgenes. As a control, RNA was harvested from transiently-transfected HEK293T cells expressing the individual lentiviruses. Total RNA was isolated using TRIzol reagent (Invitrogen), treated with DNase I and purified (RNeasy Plus kit, Qiagen) before synthesis of first-strand cDNA using the SuperScript III First-Strand Synthesis System (Invitrogen). Quantitative PCR was performed on three technical replicates using the RT-real time SYBR green PCR Mastermix (SA Biosciences) and primers that specifically amplify the proviral encoded transgenes. Lentiviral-specific primers consist of a gene-specific sense primer and a common antisense primer located downstream of each transgene within the proviral backbone. Sense primers: Oct4 5′-TCTGTTCCCGT CACTGCTCT-3′, Sox2 5′-CGCCCAGTAGACTGCACAT-3′, cMyc 5′-TGTCCATTCAAGCAGACG AG-3′, Klf4 5′-CACTACCGCAAACACACAGG-3′. Common antisense primer 5′-GGCATTAAAGCAGCGTATCC-3′. PCR conditions were 94° C. for 4 min, 40 cycles of 94° C. for 30 s, 55° C. for 30 s, 72° C. for 30 s. Data was generated on a MJ Research Chromo4 PTC-200 thermal cycler and extracted with Opticon Monitor software.

Transgene expression level for iPSCs was normalized to Gapdh expression (Gapdh forward 5′-TCAACGGGAAGCCCATCA-3′, Gapdh reverse 5′-CTCGTGGTTCACACCCATCA-3′) and plotted relative to transgenes expressed in transfected HEKs. The Gapdh primer pair did not amplify human GAPDH efficiently; therefore we normalized HEK293T transgene expression values to the average Gapdh expression value for iPSCs. It is important to note that while this analysis produces accurate relative expression levels for the same gene across various iPSC lines, it provides only a rough estimate of the relative expression levels between transfected HEK293T cells and the iPSCs and this should not be considered quantitative.

When lentiviral expression was re-induced with dox, iPSC lines tended to have one order of magnitude higher expression levels indicating that the rtTAM2.2 proviral insertion was not completely silenced in the iPSC lines and suggesting, by inference, that employing a dox inducible system can result in less residual transgene expression than non-inducible lentiviral strategies.

Generation of chimeras. Chimeras were produced by injection of iPSCs (passage 5-8) into diploid blastocysts, generated by mating superovulated C57BL/6J females to C57BL/6J×DBA2 F1 stud males, according to the standard protocol³³.

Generation of iPS fetuses and mice. For tetraploid complementation, superovulated albino (BALB/cByJ×C57BL/6J-Tyr^{c-2J}/J)F1 females were mated with males of the same hybrid strain background. One-cell embryos were collected and cultured overnight in KSOM-AA medium (Millipore). The next day tetraploid embryos were generated by blastomere electrofusion of two-cell embryos according to standard procedures and cultured under the same conditions^{33, 34}. Forty-eight hours later, tetraploid blastocysts were injected with 10-12 iPSCs each and transferred to the uterine horns of pseudopregnant recipients. IPSC-derived fetuses were dissected from the uterine horns of recipient mice at different stages of development or live newborn pups were recovered by C/section at E18.5 and fostered to CD-1 female mice.
1. Takahashi, K. et al. Induction of pluripotent stem cells from adult human fibroblasts by defined factors. Cell 131, 861-872 (2007).
2. Takahashi, K. & Yamanaka, S. Induction of pluripotent stem cells from mouse embryonic and adult fibroblast cultures by defined factors. Cell 126, 663-676 (2006).
3. Wernig, M. et al. In vitro reprogramming of fibroblasts into a pluripotent ES-cell-like state. Nature 448, 318-324 (2007).
4. Park, I. H., Lerou, P. H., Zhao, R., Huo, H. & Daley, G. Q. Generation of human-induced pluripotent stem cells. Nat Protoc 3, 1180-1186 (2008).
5. Yu, J. et al. Induced pluripotent stem cell lines derived from human somatic cells. Science 318, 1917-1920 (2007).
6. Maherali, N. et al. Directly reprogrammed fibroblasts show global epigenetic remodeling and widespread tissue contribution. Cell Stem Cell 1, 55-70 (2007).
7. Zhou, H. et al. Generation of Induced Pluripotent Stem Cells Using Recombinant Proteins. Cell Stem Cell (2009).
8. Ebert, A. D. et al. Induced pluripotent stem cells from a spinal muscular atrophy patient. Nature 457, 277-280 (2009).
9. Park, I. H. et al. Disease-specific induced pluripotent stem cells. Cell 134, 877-886 (2008).
10. Dimos, J. T. et al. Induced pluripotent stem cells generated from patients with ALS can be differentiated into motor neurons. Science 321, 1218-1221 (2008).
11. Hanna, J. et al. Direct reprogramming of terminally differentiated mature B lymphocytes to pluripotency. Cell 133, 250-264 (2008).
12. Eggan, K. et al. Mice cloned from olfactory sensory neurons. Nature 428, 44-49 (2004).
13. Wakayama, T., Perry, A. C., Zuccotti, M., Johnson, K. R. & Yanagimachi, R. Full-term development of mice from enucleated oocytes injected with cumulus cell nuclei. Nature 394, 369-374 (1998).
14. Nagy, A., Rossant, J., Nagy, R., Abramow-Newerly, W. & Roder, J. C. Derivation of completely cell culture-derived mice from early-passage embryonic stem cells. Proc Natl Acad Sci USA 90, 8424-8428 (1993).
15. Eggan, K. & Jaenisch, R. Generation of embryonic stem (ES) cell-derived embryos and mice by tetraploid-embryo complementation. (Springer, Heidelberg; 2006).
16. Chin, M. H. et al. Induced pluripotent stem cells and embryonic stem cells are distinguished by gene expression signatures. Cell Stem Cell 5, 111-123 (2009).
17. Anton Novak, C. G., Wenyi Yang, Andras Nagy, Corrinne G. Lobe Z/EG, a double reporter mouse line that expresses enhanced green fluorescent protein upon cre-mediated excision. Genesis 28, 147-155 (2000).
18. Yumiko Nagai, H. S., Mineto Yokoi Transgenic expression of Cre recombinase in mitral/tufted cells of the olfactory bulb. genesis 43, 12-16 (2005).
19. Lois, C., Hong, E. J., Pease, S., Brown, E. J. & Baltimore, D. Germline Transmission and Tissue-Specific Expression of Transgenes Delivered by Lentiviral Vectors. Science 295, 868-872 (2002).
20. Go, W. Y., Ho, S. N. Optimization and direct comparison of the dimerizer and reverse tet transcriptional control systems. The Journal of Gene Medicine 4, 258-270 (2002).
21. Yusa, K., Rad, R., Takeda, J. & Bradley, A. Generation of transgene-free induced pluripotent mouse stem cells by the piggyBac transposon. Nat Methods 6, 363-369 (2009).
22. Huangfu, D. et al. Induction of pluripotent stem cells by defined factors is greatly improved by small-molecule compounds. Nat Biotechnol 26, 795-797 (2008).
23. Yamanaka, S. Elite and stochastic models for induced pluripotent stem cell generation. Nature 460, 49-52 (2009).
24. Mikkelsen, T. S. et al. Dissecting direct reprogramming through integrative genomic analysis. Nature 454, 49-55 (2008).
25. Eakin, G. S., Hadjantonakis, A. K., Papaioannou, V. E. & Behringer, R. R. Developmental potential and behavior of tetraploid cells in the mouse embryo. Dev Biol 288, 150-159 (2005).
26. Eggan, K. & Jaenisch, R. Differentiation of F1 embryonic stem cells into viable male and female mice by tetraploid embryo complementation. Methods Enzymol 365, 25-39 (2003).
27. Mackay, G. E. & West, J. D. Fate of tetraploid cells in 4n<-->2n chimeric mouse blastocysts. Mech Dev 122, 1266-1281 (2005).
28. Goebbels, S. et al. Genetic targeting of principal neurons in neocortex and hippocampus of NEX-Cre mice. Genesis 44, 611-621 (2006).
29. Wu, S. X. et al. Pyramidal neurons of upper cortical layers generated by NEX-positive progenitor cells in the subventricular zone. Proc Natl Acad Sci USA 102, 17172-17177 (2005).
30. Lois, C., Hong, E. J., Pease, S., Brown, E. J. & Baltimore, D. Germline Transmission and Tissue-Specific Expression of Transgenes Delivered by Lentiviral Vectors. Science 295, 868-872 (2002).
31. Xu, J., Mashimo, T., Sudhof, T C. Synaptotagmin-1, -2, and -9: Ca2+ Sensors for Fast Release that Specify Distinct Presynaptic Properties in Subsets of Neurons. Neuron 54, 567-581 (2007).
32. Go, W. Y., Ho, S. N. Optimization and direct comparison of the dimerizer and reverse tet transcriptional control systems. The Journal of Gene Medicine 4, 258-270 (2002).
33. Nagy, A., Gertsenstein, M., Vintersten, K. & Behringer, R. Manipulating the mouse embryo: A laboratory manual. (Cold Spring Harbor Laboratory Press, New York; 2003).
34. Eggan, K. & Jaenisch, R. Generation of embryonic stem (ES) cell-derived embryos and mice by tetraploid-embryo complementation. (Springer, Heidelberg; 2006).

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### Informal Sequence Listing

rtTAM2.2 amino acid sequence
SEQ ID NO: 1
MSRLDKSKVINGALELLNGVGIEGLTTRKLAQKLGVEQPTLYWHVKNK
RALLDALPIEMLDRHHTHFCPLEGESWQDFLRNNAKSFRCALLSHRDG
AKVHLGTRPTEKQYETLENQLAFLCQQGFSLENALYALSAVGHFTLGC
VLEEQEHQVAKEERETPTTDSMPPLLRQAIELFDRQGAEPAFLFGLEL
IICGLEKQLKCESGGGSLEGPPQSLSAPVPKSTQAGEGTLSEALLHLQ
FDADEDLGALLGNSTDPGVFTDLASVDNSEFQQLLNQGVSMSHSTAEP
MLMEYPEAITRLVTGSQRPPDPAPTPLGTSGLPNGLSGDEDFSSIADM
DFSALLSQISSVEGFSVDTSALLDLFSPSVTVPDMSLPDLDSSLASIQ
ELLSPQEPPRPPEAENSSPDSGKQLVHYTAQPLFLLDPGSVDTGSNDL
PVLFELGEGSYFSEGDGFAEDPTISLLTGSEPPKAKDPTVSVDYPYDV
PDYALD

1. A method for inducing full pluripotency in non-pluripotent animal cells, the method comprising,
introducing one or more transcription factor expression cassette(s) into non-pluripotent animal cells, which expression cassette(s) comprise a promoter operably linked to a polynucleotide encoding one or more transcription factors sufficient to induce pluripotency into the cells, where expression of the transcription factors is controlled by an inducible element that can be induced by an inducer;
contacting the cells comprising the transcription factor expression cassette(s) and the transcriptional activator expression cassette with the inducer; and
contacting the cells with a chromatin modifier or histone deacetylase inhibitor; and
selecting cells that are pluripotent, thereby inducing pluripotency in non-pluripotent animal cells.

2. The method of claim 1, wherein the cells are contacted with (1) the inducer and (2) the chromatin modifier or histone deacetylase inhibitor for at least 13 days prior to the selecting step.

3. The method of claim 1, wherein the cells are contacted with (1) the inducer and (2) the chromatin modifier or histone deacetylase inhibitor for 13-30 days prior to the selecting step.

4. The method of claim 1, wherein the cells are contacted with (1) the inducer and (2) the chromatin modifier or histone deacetylase inhibitor for 19-30 days prior to the selecting step.

5. The method of claim 1, wherein the histone deacetylation inhibitor is valproic acid.

6. The method of claim 1, wherein the expression of the transcription factors is controlled by a tetracycline and/or doxycycline-inducible tetO regulatory element; and
the method comprises introducing a transcriptional activator expression cassette comprising a promoter operably linked to a polynucleotide encoding a tetracycline and/or doxycycline responsive transcriptional activator, wherein the transcriptional activator comprises a reverse tet repressor fused to a heterologous transactivation domain.

7.-10. (canceled)

11. The method of claim 1, wherein the one or more transcription factors comprise at least a Sox polypeptide and an Oct3/4 polypeptide.

12. The method of claim 1, wherein the one or more transcription factors comprise Oct4, Sox2, Klf4, and c-Myc.

13.-70. (canceled)

71. A method for inducing full pluripotency in non-pluripotent animal cells, the method comprising,
introducing one or more transcription factor expression cassette(s) into non-pluripotent animal cells, which expression cassette(s) comprise a promoter operably linked to a polynucleotide encoding one or more transcription factors sufficient to induce pluripotency into the cells, wherein the expression cassettes are inserted into the genome of the cell in no more than 1, 2, or 3 copies, and wherein the transcription factor expression cassettes are under control of an operator responsive to a transcriptional activator; and
introducing a transcriptional activator expression cassette comprising a promoter operably linked to a polynucleotide encoding the transcriptional activator, wherein the transcriptional activator activates expression from the transcription factor expression cassettes more than if a rtTAM2 transcriptional activator were used;
inducing activation of the transcriptional activator, if necessary; and
selecting cells that are pluripotent, thereby inducing pluripotency in non-pluripotent animal cells.

72. The method of claim 71, wherein the inducing step lasts at least 13 days prior to the selecting step.

73. The method of claim 71, wherein the inducing step lasts for 19-30 days prior to the selecting step.

74.-87. (canceled)

88. An isolated non-embryonic animal cell or cell line or cell culture, wherein the cell is capable of generating an adult animal.

89. The cell, cell line or cell culture of claim 88, wherein the animal is a mouse.

90. The cell, cell line or cell culture of claim 88, wherein the animal is a non-human animal.

91. The cell, cell line or cell culture of claim 88, wherein the animal is a human.

92. The cell, cell line or cell culture of claim 88, wherein the cells have an appropriate imprinting at the Dlk1-Gtl2 locus to allow for expression of RNA from the locus.

93. (canceled)

94. An isolated cell selected by the method of claim 1.


SEQUENCE LISTING (12 sequences)

SEQ ID NO: 1 - protein, 486 aa, Artificial Sequence
  synthetic reverse tet transactivator rtTAM2.2, heterologous fusion transactivation domain
MSRLDKSKVINGALELLNGVGIEGLTTRKLAQKLGVEQPTLYWHVKNKRALLDALPIEMLDRHHTHFCPLEGESWQDFLRNNAKSFRCALLSHRDGAKVHLGTRPTEKQYETLENQLAFLCQQGFSLENALYALSAVGHFTLGCVLEEQEHQVAKEERETPTTDSMPPLLRQAIELFDRQGAEPAFLFGLELIICGLEKQLKCESGGGSLEGPPQSLSAPVPKSTQAGEGTLSEALLHLQFDADEDLGALLGNSTDPGVFTDLASVDNSEFQQLLNQGVSMSHSTAEPMLMEYPEAITRLVTGSQRPPDPAPTPLGTSGLPNGLSGDEDFSSIADMDFSALLSQISSVEGFSVDTSALLDLFSPSVTVPDMSLPDLDSSLASIQELLSPQEPPRPPEAENSSPDSGKQLVHYTAQPLFLLDPGSVDTGSNDLPVLFELGEGSYFSEGDGFAEDPTISLLTGSEPPKAKDPTVSVDYPYDVPDYALD

SEQ ID NO: 2 - DNA, 19 bp, Artificial Sequence
  synthetic microsatellite PCR assay D12Mit136 sense primer
tttaattttgagtgggttt

SEQ ID NO: 3 - DNA, 24 bp, Artificial Sequence
  synthetic microsatellite PCR assay D12Mit136 antisense primer
tggctacatgtacactgatctcca

SEQ ID NO: 4 - DNA, 19 bp, Artificial Sequence
  synthetic albino allele PCR assay sense primer specific for c-2JTyr mutation
tcaaaggggtggatgacct

SEQ ID NO: 5 - DNA, 20 bp, Artificial Sequence
  synthetic albino allele PCR assay antisense primer
cccccaaatccaaacttaca

SEQ ID NO: 6 - DNA, 20 bp, Artificial Sequence
  synthetic RT-qPCR lentiviral gene-specific sense primer Oct4
tctgttcccgtcactgctct

SEQ ID NO: 7 - DNA, 19 bp, Artificial Sequence
  synthetic RT-qPCR lentiviral gene-specific sense primer Sox2
cgcccagtagactgcacat

SEQ ID NO: 8 - DNA, 18 bp, Artificial Sequence
  synthetic RT-qPCR lentiviral gene-specific sense primer cMyc
tgtccattcaagcagacg

SEQ ID NO: 9 - DNA, 20 bp, Artificial Sequence
  synthetic RT-qPCR lentiviral gene-specific sense primer Klf4
cactaccgcaaacacacagg

SEQ ID NO: 10 - DNA, 20 bp, Artificial Sequence
  synthetic RT-qPCR common antisense primer
ggcattaaagcagcgtatcc

SEQ ID NO: 11 - DNA, 18 bp, Artificial Sequence
  synthetic Gapdh forward primer
tcaacgggaagcccatca

SEQ ID NO: 12 - DNA, 20 bp, Artificial Sequence
  synthetic Gapdh reverse primer
ctcgtggttcacacccatca